(19) United States
(12) Patent Application Publication
(10) Pub. No.: US 2025/0041605 A1
(43) Pub. Date: Feb. 6, 2025

(54) SYSTEMS AND METHODS FOR NEUROMODULATION BASED ON SELECTIVE EVOKED RESPONSES

(71) Applicant: BOSTON Scientific Neuromodulation Corporation, Valencia, CA (US)

(72) Inventors: G. Karl Steinke, Valencia, CA (US); Tomasz Mark Fraczek, Yucaipa, CA (US)

(21) Appl. No.: 18/784,365

(22) Filed: Jul. 25, 2024

Related U.S. Application Data
(60) Provisional application No. 63/529,950, filed on Jul. 31, 2023.

(51) Int. Cl. A61N 1/36; A61N 1/05; A61N 1/372; A61N 1/375; A61N 1/378

(52) U.S. Cl. CPC A61N 1/36135; A61N 1/0534; A61N 1/36185; A61N 1/37247; A61N 1/3754; A61N 1/378

(57) ABSTRACT

Systems and methods for selective sensing of evoked responses (ERs) and using the ERs to guide neuromodulation are disclosed. An exemplary system comprises at least one multi-electrode lead, an electrostimulator to provide electrostimulation to a neural target, a sensing circuit to sense ERs to electrostimulation, and a controller circuit. In response to electrostimulation delivered to the neural target in accordance with a stimulation setting via a stimulating electrode, the controller circuit can collect ERs from each of a group of sensing electrodes selected from and less than an entirety of the electrodes on the lead. The selected electrodes can be distinct from the stimulating electrode, or within a specific proximity to the stimulating electrode. The controller circuit can use a comparison of the sensed ERs to acceptance criterion to aid in lead placement and stimulation programming.

### CLAIM OF PRIORITY

This application claims the benefit of U.S. Provisional Application No. 63/529,950 filed on Jul. 31, 2023, which is hereby incorporated by reference in its entirety.

### TECHNICAL FIELD

This document relates generally to medical systems, and more particularly, but not by way of limitation, to systems, devices, and methods for selective sensing of evoked response and using the same to guide neurostimulation therapy.

### BACKGROUND

Medical devices may include therapy-delivery devices configured to deliver a therapy to a patient and/or monitors configured to monitor a patient condition via user input and/or sensor(s). For example, therapy-delivery devices for ambulatory patients may include wearable devices and implantable devices, and further may include, but are not limited to, stimulators (such as electrical, thermal, or mechanical stimulators) and drug delivery devices (such as an insulin pump). An example of a wearable device includes, but is not limited to, transcutaneous electrical neural stimulators (TENS), such as may be attached to glasses, an article of clothing, or a patch configured to be adhered to skin. Implantable stimulation devices may deliver electrical stimuli to treat various biological disorders, such as pacemakers to treat cardiac arrhythmia, defibrillators to treat cardiac fibrillation, heart failure cardiac resynchronization therapy devices, cochlear stimulators to treat deafness, retinal stimulators to treat blindness, muscle stimulators to produce coordinated limb movement, spinal cord stimulators (SCS) to treat chronic pain, cortical and Deep Brain Stimulators (DBS) to treat motor and psychological disorders, Peripheral Nerve Stimulation (PNS), Functional Electrical Stimulation (FES), and other neural stimulators to treat urinary incontinence, sleep apnea, shoulder subluxation, etc. A neurostimulation device (e.g., DBS, SCS, PNS or TENS) may be configured to treat pain. By way of example and not limitation, a DBS system may be configured to treat tremor, bradykinesia, and dyskinesia and other motor disorders associated with Parkinson's Disease (PD).

It has been proposed to use evoked potentials (also referred to as evoked responses or ERs) to guide neurostimulation therapy. For example, Evoked Resonant Neural Activity (ERNA) has been proposed as a feedback signal for subthalamic nucleus (STN) DBS therapy for Parkinson's disease. ERNA may also be referred to by other names such as DBS Local Evoked Potentials (DLEP), Evoked oscillatory neural responses (EONR), and other terms. Evoked potentials, including ERNA, may be present in other indications and anatomical structures or locations.

It is desired to improve lead placement and/or programming a neurostimulator based on ERs such as ERNA while reducing the time and effort required for identifying the lead position and/or the programming setting that would produce the desired or target response.

### SUMMARY

The present inventors have recognized that the desired evoked response target varies. For example, the ERNA target for STN DBS may vary among patients. The present inventors recognize that the ERNA target, or other evoked response target, may vary based on anatomical target and trajectory of the lead placement and can vary with other factors such as the evoking and recording settings, the surgery center, the surgeon, the surgical or programming techniques, preferences, and errors. To accommodate the variations in desired evoked response targets, multiple stimulation-ER tests may be performed, and a large volume of ER recordings may be collected from multiple locations via respective sensing electrodes (also referred to as recording electrodes). The ERs may be analyzed to determine if the measured ERs match a desired or target ER response. Such decision may be used to guide lead placement, such as pushing, pulling, shifting, or rotating the lead to modify lead trajectory or lead depth, thereby facilitating identification of a desired stimulation location that maximally reduces symptoms while inducing minimal side effect. Such stimulation effect can be represented by a desired target evoked response. However, lead placement and/or programming optimization can be time consuming and take a lot of system resources. Embodiments of the present subject matter provide systems, device and methods that implement sensing ERs from a selected group of electrodes to improve the efficiency without compromising the accuracy of identifying ERs that match the desired or target response for different desired evoked response targets. The selected sensing electrodes can be distinct from the stimulating electrode(s) delivering the electrostimulation energy, or within a specific proximity to the stimulating electrode. A model-based artifact characterization and removal technique can improve the quality of ERs sensed from the selected group of electrodes, produce a more robust estimate of ER distribution, and facilitate a more reliable search for a match to the desired or target response.

An example (e.g., “Example 1”) of a medical-device system may include: at least one lead including a plurality of electrodes; an electrostimulator configured to provide electrostimulation to a neural target of a patient; a sensing circuit configured to sense an evoked response (ER) to the electrostimulation; and a controller circuit operably connected to the electrostimulator and the sensing circuit, the controller circuit configured to: deliver the electrostimulation to the neural target in accordance with a stimulation setting via a stimulating electrode selected from the plurality of electrodes on the at least one lead; collect sensed ERs to the electrostimulation from each of a group of sensing electrodes selected from, and less than an entirety of, the plurality of electrodes on the at least one lead, the group of selected sensing electrodes located within a specific proximity to the selected stimulating electrode; compare the ERs sensed from the group of selected sensing electrodes to an acceptance criterion to produce a comparison result; and display the ERs and the comparison result on a user interface.

In Example 2, the subject matter of Example 1 optionally includes the selected group of sensing electrodes that may include two or more electrodes immediate adjacent to the stimulating electrode on the at least one lead.

In Example 3, the subject matter of any one or more of Examples 1-2 optionally includes the controller circuit that may be further configured to, based at least in part on the comparison result, provide a recommendation on the user interface to reposition the at least one lead or to adjust the stimulation setting to cause the sensed ERs from the group of selected sensing electrodes to compare more favorably to the acceptance criterion.

In Example 4, the subject matter of any one or more of Examples 1-3 optionally includes the at least one lead that may include a deep brain stimulation (DBS) lead, and wherein the electrostimulator is configured to provide DBS to a brain target of the patient.

In Example 5, the subject matter of any one or more of Examples 1˜4 optionally includes the plurality of electrodes that may include one or more ring electrodes disposed at respective longitudinal positions along a length of the at least one lead, or one or more rows of segmented electrodes where each row comprises segmented electrodes disposed about a circumference of the at least one lead at a specific longitudinal position, wherein the stimulating electrode and the group of selected sensing electrodes are each selected from the one or more ring electrodes or the one or more rows of segmented electrodes.

In Example 6, the subject matter of any one or more of Examples 1-5 optionally includes the controller circuit that can be configured to: during each of multiple stimulation sessions, deliver electrostimulation in accordance with respective stimulation settings via respective stimulating electrodes, and collect sensed ERs to the electrostimulation from respective groups of selected sensing electrodes; accumulate the sensed ERs collected from the multiple stimulation sessions; and compare the accumulated ERs to the acceptance criterion to produce the comparison result.

In Example 7, the subject matter of any one or more of Examples 1-6 optionally includes the acceptance criterion that may include acceptance bounds for the ERs sensed from the group of selected sensing electrodes.

In Example 8, the subject matter of any one or more of Examples 1-7 optionally includes the acceptance criterion that may include a target ER template representing a patient-specific ER or a population-based ER to electrostimulation of the neural target.

In Example 9, the subject matter of Example 8 optionally includes the target ER template that may include a target distribution of ERs across the group of selected sensing electrodes, wherein the controller circuit is configured to: determine a spatial distribution of the sensed ERs across the group of selected sensing electrodes; and provide a recommendation on the user interface to reposition the at least one lead or to adjust the stimulation setting based at least in part on a comparison of the determined spatial distribution of the sensed ERs and the target distribution.

In Example 10, the subject matter of any one or more of Examples 8-9 optionally includes the target ER template that may include a target ER feature, wherein the controller circuit is configured to: determine an ER feature from the ERs sensed from the group of selected sensing electrodes; and provide a recommendation on the user interface to reposition the at least one lead or to adjust the stimulation setting based at least in part on a comparison of the determined ER feature to the target ER feature.

In Example 11, the subject matter of any one or more of Examples 1-10 optionally includes the controller circuit that may be further configured to filter the ERs sensed from the group of selected sensing electrodes to remove or substantially attenuate an artifact component from each of the ERs, and to compare the filtered ERs to the acceptance criterion to produce the comparison result.

In Example 12, the subject matter of Example 11 optionally includes the controller circuit that may be configured to, for each of the ERs: generate a parametric model to fit the artifact component in the each of the ERs in accordance with a fitting criterion; and subtract the parametric model fitted artifact component from the each of the ERs.

In Example 13, the subject matter of Example 12 optionally includes the parametric model that may be a polynomial-exponential decay model.

In Example 14, the subject matter of any one or more of Examples 11-13 optionally includes the controller circuit that may be configured to, for each of the ERs: generate a time-reversed signal for each of the ERs; generate a parametric model to fit the artifact component in the time-reversed signal in accordance with a fitting criterion; and subtract the parametric model fitted artifact component from the time-reversed signal.

In Example 15, the subject matter of any one or more of Examples 1-14 optionally includes the controller circuit that may be configured to intermittently pause delivery of the electrostimulation, and to collect the ERs sensed from the group of selected sensing electrodes during the intermittent pause.

Example 16 is a method of providing neurostimulation to a neural target of a patient via a medical-device system that comprises an electrostimulator and at least one lead coupled thereto, the method comprising: delivering electrostimulation to the neural target in accordance with a stimulation setting via a stimulating electrode selected from a plurality of electrodes on the at least one lead; sensing evoked responses (ERs) from each of a group of sensing electrodes electrically connected to a sensing circuit, the group of sensing electrodes selected from, and less than an entirety of, the plurality of electrodes on the at least one lead, and located within a specific proximity to the selected stimulating electrode; comparing the ERs sensed from the group of selected sensing electrodes to an acceptance criterion to produce a comparison result; displaying the ERs and the comparison result on a user interface; and based at least in part on the comparison result, providing a recommendation on the user interface to reposition the at least one lead or to adjust the stimulation setting to cause the sensed ERs from the group of selected sensing electrodes to compare more favorably to the acceptance criterion.

In Example 17, the subject matter of Example 16 optionally includes the selected group of sensing electrodes that may include two or more electrodes immediate adjacent to the stimulating electrode on the at least one lead.

In Example 18, the subject matter of any one or more of Examples 16-17 optionally includes determining a spatial distribution of the sensed ERs across the group of selected sensing electrodes, wherein the recommendation to reposition the at least one lead or to adjust the stimulation setting is based at least in part on a comparison of the determined spatial distribution of the sensed ERs and a target distribution of ERs across the group of selected sensing electrodes.

In Example 19, the subject matter of any one or more of Examples 16-18 optionally includes determining an ER feature from the ERs sensed from the group of selected sensing electrodes, wherein the recommendation to reposition the at least one lead or to adjust the stimulation setting is based at least in part on a comparison of the determined ER feature to a target ER feature.

In Example 20, the subject matter of any one or more of Examples 16-19 optionally includes filtering the ERs sensed from the group of selected sensing electrodes to remove or substantially attenuate an artifact component from each of the ERs, and comparing the filtered ERs to the acceptance criterion to produce the comparison result.

In Example 21, the subject matter of Example 20 optionally includes filtering the ERs sensed from the group of selected sensing electrodes that may include, for each of the ERs: generating a parametric model to fit the artifact component in the each of the ERs in accordance with a fitting criterion; and subtracting the parametric model fitted artifact component from the each of the ERs.

In Example 22, the subject matter of Example 21 optionally includes the parametric model that may be a polynomial-exponential decay model.

In Example 23, the subject matter of any one or more of Examples 20-22 optionally includes filtering the ERs sensed from the group of selected sensing electrodes includes, for each of the ERs: generating a time-reversed signal for each of the ERs; generating a parametric model to fit the artifact component in the time-reversed signal in accordance with a fitting criterion; and subtracting the parametric model fitted artifact component from the time-reversed signal.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates, by way of example and not limitation, an electrical stimulation system, which may be used to deliver DBS.

FIG. 2 illustrates, by way of example and not limitation, an implantable pulse generator (IPG) in a DBS system.

FIGS. 3A-3B illustrate, by way of example and not limitation, leads that may be coupled to the IPG to deliver electrostimulation such as DBS.

FIG. 4 illustrates, by way of example and not limitation, a computing device for programming or controlling the operation of an electrical stimulation system.

FIG. 5 illustrates, by way of example, an example of an electrical therapy-delivery system.

FIG. 6 illustrates, by way of example and not limitation, a monitoring system and/or the electrical therapy-delivery system of FIG. 5, implemented using an implantable medical device (IMD).

FIG. 7 illustrates an example of a neuromodulation system configured to provide guided implantation (e.g., lead placement) and device programming based on selected evoked responses.

FIG. 8 illustrates an example of a stimulating-sensing electrode configuration graphically represented by an ER sensing map.

FIGS. 9A-9D illustrate an example of stimulating-sensing electrode configuration that involves a selected subset, less than an entirety, of available electrodes on a lead for sensing ER.

FIG. 10 illustrates an example of an ER sensing map where the ERs are sensed from nearest neighbor electrodes with respect to the stimulating electrode.

FIGS. 11A-11D illustrate example effects of filtering the ERs sensed from nearest neighbor electrodes to remove or substantially attenuate stimulation artifacts modeled by a polynomial-exponential decay model.

FIGS. 12A-12D illustrate example effects of filtering time-reversed version of ERs sensed from nearest neighbor electrodes to remove or substantially attenuate stimulation artifacts modeled by a polynomial-exponential decay model.

FIG. 13 illustrates, by way of example and not limitation, a display for user interface that creates a response fit from a distribution of ERs.

FIG. 14 illustrates, by way of example and not limitation, embodiments for measuring and matching ERs, or ER features or a distribution of ER features derived therefrom, to an acceptance criterion such as a template.

FIG. 15 illustrates, by way of example and not limitation, ER distributions for DBS lead electrodes.

FIG. 16 illustrates, by way of example and not limitation, distributions of ERs sensed respectively from distinct neural targets in left and right hemispheres of a brain.

FIG. 17 illustrates, by way of example and not limitation, a method of guided implantation (e.g., lead placement) and device programming using selected ERs.

FIG. 18 illustrates generally a block diagram of an example machine upon which any one or more of the techniques (e.g., methodologies) discussed herein may perform.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

There is no single universal “optimal” stimulation target and desired response because the optimal target may vary depending upon condition or disorder, anatomy (anatomical target), lead and trajectory, center (imaging equipment position, etc.), surgeon (how they implant and position lead, their preferences for lead placement), or symptoms to improve (e.g., tremor to cognitive skill improvement). Raw responses may be displayed and the user may determine fitness based on the displayed raw responses. The user may adjust feature target bounds, acceptance thresholds and maximum number of steps before convergence.

Evoked responses (ERs) may be used to guide an implantation procedure (e.g., lead placement or electrode positioning), or programming of device settings (e.g., sensing parameters or neurostimulation parameters). The ERs may be caused by stimulation that provides ERs, stimulation that provides therapy and stimulation that can both deliver therapy and provide ERs. Stimulation may be located (1) where placing evoking pulses gets a desired response such as to maximize ERNA, (2) where listening for responses gets a desired response (e.g., maximize ERNA), (3) where placing lead is desired (e.g., best for therapy, and (4) where placing stimulation on the lead is desired (e.g., maximize therapy and/or minimize/counter side effects). Responses may be modulated by the details of the sensing, including amplifier settings, relationships between stimulating and sensing electrodes, natures of stimulating or sensing electrodes including geometry and surface among other factors, and signal processing occurring during and after measurement, including treatment within analogue or digital hardware, firmware, or software. The target may be an anatomical targe such as like a volume (e.g., STN), a collection of fibers, a sub-region (motor STN; dorsolateral STN), a volume of interest described within or related to a particular patient's brain such as from atlas or aggregate prior information, or a “point” that may be described by optimizing a stimulation location (e.g., one of (1)-(4) above).

Various embodiments as described in this document may implement sensed ERs from a selected group of sensing electrodes to improve the efficiency of searching for ERs that match the desired or target response for different desired evoked response targets. Conventionally, multiple stimulation-ER tests may be required to produce sufficient ER recordings that can be used to characterize a spatial distribution of tissue responses to electrostimulation. The tests generally involve repeated recordings of ERS from a large array of sensing electrodes placed at respective sensing locations in response to stimulation from a stimulating electrode with varying stimulation settings (e.g., amplitude, frequency, or pulse width). In some cases, a stimulating electrode may also sense ER to stimulation delivered from the same stimulating electrode. When stimulating electrodes and sensing electrodes are depicted on a two-dimensional array, the ERs include “on-diagonal” ERs sensed by the same stimulating electrodes, and “off-diagonal” ERs sensed by electrodes different than the stimulating electrode. Although the “on-diagonal” ERs at the stimulation site may provide useful information about ER distribution and therefore for identifying a match to the desired or target response, the “on-diagonal” ERs are prone to stimulation artifacts strong enough to contaminate the ER component of interest. In addition, some “off-diagonal” ERs particularly those from sensing electrodes in close proximity of the stimulating electrode may also be contaminated by stimulation artifacts. The artifacts may also include DC offsets, time-dependent offsets, components independent of stimulation. The stimulation artifacts are conventionally managed using hardware components and techniques such as analog filters and digital-to-analog converters (DACs) with sufficiently wide active range and enough steps. The hardware, however, can be large and the settings are usually inconvenient to adjust. The present document describes systems and methods for sensing ERs from a selected group of electrodes to improve the efficiency without compromising the accuracy of identifying ERs that match the desired or target response for different desired evoked response targets. The selected sensing electrodes can be distinct from the stimulating electrode, or within a specific proximity to, and having a particular geometric relationship with the stimulating electrode. Additionally, various embodiments implement a model-based artifact characterization and removal technique to improve signal quality of ERs sensed from the selected group of electrodes, and to produce a more robust estimate of ER distribution and a more reliable search for a match to the desired or target response. The “off-diagonal” ERs particularly those from sensing electrodes in close proximity of the stimulating electrode and the model-based artifact characterization and removal as described herein may be used in various steps involved in device implantation and stimulation programming process including, for example, lead trajectory confirmation (during implantation), lead depth confirmation (during implantation), stimulation and sensing electrode selection (during clinical programming), etc.

This disclosure refers to an ERNA target for DBS, such as may be used to treat Parkinson's Disease, as a nonlimiting example of an ER to electrostimulation provided by an electrostimulator. The present subject matter may be applied for other ERs to other electrostimulation. The electrostimulation may be therapeutic in nature in some examples, or diagnostic in nature in others.

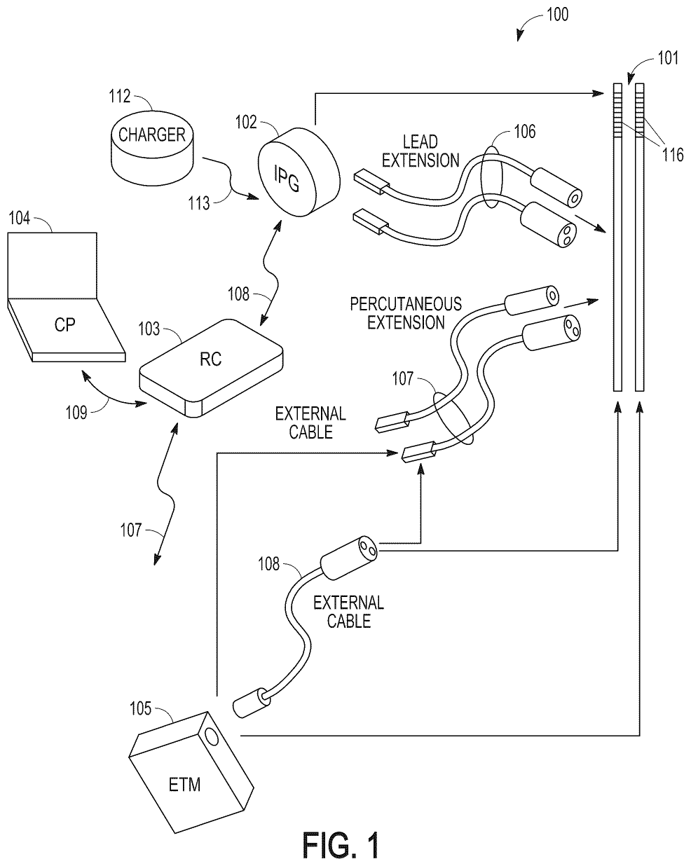

FIG. 1 illustrates, by way of example and not limitation, an electrical stimulation system 100, which may be used to deliver DBS. The electrical stimulation system 100 may generally include a one or more (illustrated as two) of implantable neuromodulation leads 101, a waveform generator such as an implantable pulse generator (IPG) 102, an external remote controller (RC) 103, a clinician programmer (CP) 104, and an external trial modulator (ETM) 105. The IPG 102 may be physically connected via one or more percutaneous lead extensions 106 to the neuromodulation lead(s) 101, which carry a plurality of electrodes 116. The electrodes, when implanted in a patient, form an electrode arrangement. As illustrated, the neuromodulation leads 101 may be percutaneous leads with the electrodes arranged in-line along the neuromodulation leads or about a circumference of the neuromodulation leads. Any suitable number of neuromodulation leads can be provided, including only one, as long as the number of electrodes is greater than two (including the IPG case function as a case electrode) to allow for lateral steering of the current. Alternatively, a surgical paddle lead can be used in place of one or more of the percutaneous leads. The IPG 102 includes pulse generation circuitry that delivers electrical modulation energy in the form of a pulsed electrical waveform (i.e., a temporal series of electrical pulses) to the electrodes in accordance with a set of modulation parameters.

The ETM 105 may also be physically connected via the percutaneous lead extensions 107 and external cable 108 to the neuromodulation lead(s) 101. The ETM 105 may have similar pulse generation circuitry as the IPG 102 to deliver electrical modulation energy to the electrodes in accordance with a set of modulation parameters. The ETM 105 is a non-implantable device that may be used on a trial basis after the neuromodulation leads 101 have been implanted and prior to implantation of the IPG 102, to test the responsiveness of the modulation that is to be provided. Functions described herein with respect to the IPG 102 can likewise be performed with respect to the ETM 105.

The RC 103 may be used to telemetrically control the ETM 105 via a bi-directional RF communications link 109. The RC 103 may be used to telemetrically control the IPG 102 via a bi-directional RF communications link 110. Such control allows the IPG 102 to be turned on or off and to be programmed with different modulation parameter sets. The IPG 102 may also be operated to modify the programmed modulation parameters to actively control the characteristics of the electrical modulation energy output by the IPG 102. A clinician may use the CP 104 to program modulation parameters into the IPG 102 and ETM 105 in the operating room and in follow-up sessions.

The CP 104 may indirectly communicate with the IPG 102 or ETM 105, through the RC 103, via an IR communications link 111 or another link. The CP 104 may directly communicate with the IPG 102 or ETM 105 via an RF communications link or other link (not shown). The clinician detailed modulation parameters provided by the CP 104 may also be used to program the RC 103, so that the modulation parameters can be subsequently modified by operation of the RC 103 in a stand-alone mode (i.e., without the assistance of the CP 104). Various devices may function as the CP 104. Such devices may include portable devices such as a lap-top personal computer, mini-computer, personal digital assistant (PDA), tablets, phones, or a remote control (RC) with expanded functionality. Thus, the programming methodologies can be performed by executing software instructions contained within the CP 104. Alternatively, such programming methodologies can be performed using firmware or hardware. In any event, the CP 104 may actively control the characteristics of the electrical modulation generated by the IPG 102 to allow the desired parameters to be determined based on patient feedback or other feedback and for subsequently programming the IPG 102 with the desired modulation parameters. To allow the user to perform these functions, the CP 104 may include user input device (e.g., a mouse and a keyboard), and a programming display screen housed in a case. In addition to, or in lieu of, the mouse, other directional programming devices may be used, such as a trackball, touchpad, joystick, touch screens or directional keys included as part of the keys associated with the keyboard. An external device (e.g., CP) may be programmed to provide display screen(s) that allow the clinician to, among other functions, select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical modulation energy output by the neuromodulation leads, and select and program the IPG with modulation parameters, including electrode selection, in both a surgical setting and a clinical setting. The external device(s) (e.g., CP and/or RC) may be configured to communicate with other device(s), including local device(s) and/or remote device(s). For example, wired and/or wireless communication may be used to communicate between or among the devices.

An external charger 112 may be a portable device used to transcutaneous charge the IPG 102 via a wireless link such as an inductive link 113. Once the IPG 102 has been programmed, and its power source has been charged by the external charger or otherwise replenished, the IPG 102 may function as programmed without the RC 103 or CP 104 being present.

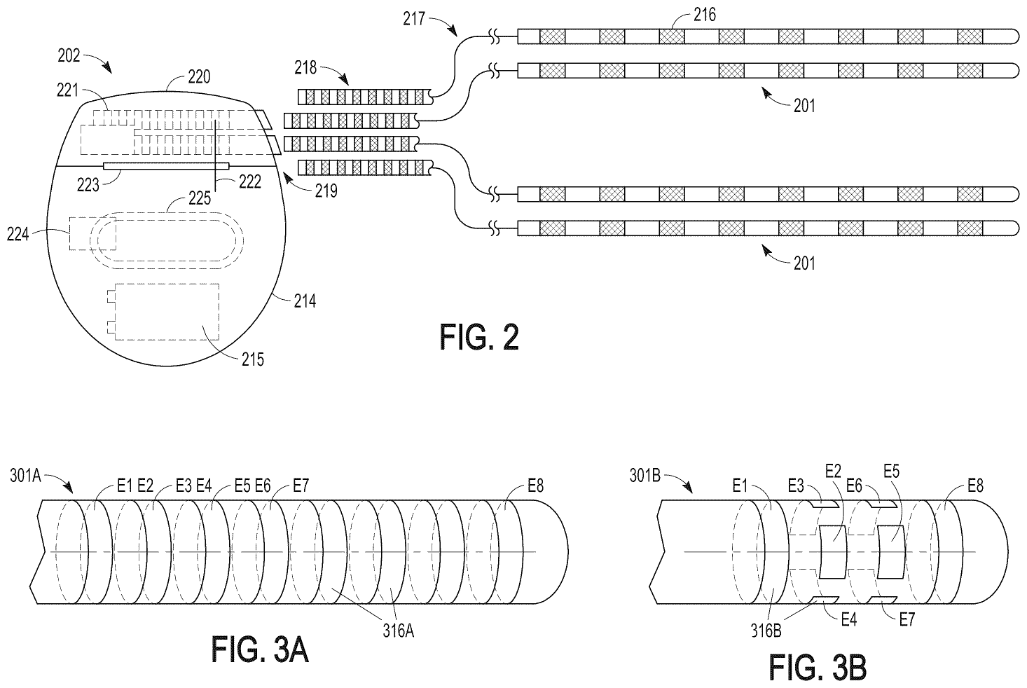

FIG. 2 illustrates, by way of example and not limitation, an IPG 202 in a DBS system. The IPG 202, which is an example of the IPG 102 of the electrical stimulation system 100 as illustrated in FIG. 1, may include a biocompatible device case 214 that holds the circuitry and a battery 215 for providing power for the IPG 202 to function, although the IPG 202 can also lack a battery and can be wirelessly powered by an external source. The IPG 202 may be coupled to one or more leads, such as leads 201 as illustrated herein. The leads 201 can each include a plurality of electrodes 216 for delivering electrostimulation energy, recording electrical signals, or both. In some examples, the leads 201can be rotatable so that the electrodes 216 can be aligned with the target neurons after the neurons have been located such as based on the recorded signals. The electrodes 216 can include one or more ring electrodes, and/or one or more rows of segmented electrodes (or any other combination of electrodes), examples of which are discussed below with reference to FIGS. 3A and 3B.

The leads 201 can be implanted near or within the desired portion of the body to be stimulated. In an example of operations for DBS, access to the desired position in the brain can be accomplished by drilling a hole in the patient's skull or cranium with a cranial drill (commonly referred to as a burr), and coagulating and incising the dura mater, or brain covering. A lead can then be inserted into the cranium and brain tissue with the assistance of a stylet (not shown). The lead can be guided to the target location within the brain using, for example, a stereotactic frame and a microdrive motor system. In some examples, the microdrive motor system can be fully or partially automatic. The microdrive motor system may be configured to perform actions such as inserting, advancing, rotating, or retracing the lead.

Lead wires 217 within the leads may be coupled to the electrodes 216 and to proximal contacts 218 insertable into lead connectors 219 fixed in a header 220 on the IPG 202, which header can comprise an epoxy for example. Alternatively, the proximal contacts 218 may connect to lead extensions (not shown) which are in turn inserted into the lead connectors 219. Once inserted, the proximal contacts 218 connect to header contacts 221 within the lead connectors 219, which are in turn coupled by feedthrough pins 222 through a case feedthrough 223 to stimulation circuitry 224 within the case 214. The type and number of leads, and the number of electrodes, in an IPG is application specific and therefore can vary.

The IPG 202 can include an antenna 225 allowing it to communicate bi-directionally with a number of external devices. The antenna 225 may be a conductive coil within the case 214, although the coil of the antenna 225 may also appear in the header 220. When the antenna 225 is configured as a coil, communication with external devices may occur using near-field magnetic induction. The IPG 202 may also include a radiofrequency (RF) antenna. The RF antenna may comprise a patch, slot, or wire, and may operate as a monopole or dipole, and preferably communicates using far-field electromagnetic waves, and may operate in accordance with any number of known RF communication standards, such as Bluetooth, Zigbee, WiFi, Medical Implant Communication System (MICS), and the like.

In a DBS application, as is useful in the treatment of tremor in Parkinson's disease for example, the IPG 202 is typically implanted under the patient's clavicle (collarbone). The leads 201 (which may be extended by lead extensions, not shown) can be tunneled through and under the neck and the scalp, with the electrodes 216 implanted through holes drilled in the skull and positioned for example in the subthalamic nucleus (STN) in each brain hemisphere. The IPG 202 can also be implanted underneath the scalp closer to the location of the electrodes' implantation. The leads 201, or the extensions, can be integrated with and permanently connected to the IPG 202 in other solutions.

Stimulation in IPG 202 is typically provided by pulses each of which may include one phase or multiple phases. For example, a monopolar stimulation current can be delivered between a lead-based electrode (e.g., one of the electrodes 216) and a case electrode. A bipolar stimulation current can be delivered between two lead-based electrodes (e.g., two of the electrodes 216). Stimulation parameters typically include current amplitude (or voltage amplitude), frequency, pulse width of the pulses or of its individual phases; electrodes selected to provide the stimulation; polarity of such selected electrodes, i.e., whether they act as anodes that source current to the tissue, or cathodes that sink current from the tissue. Each of the electrodes can either be used (an active electrode) or unused (OFF). When the electrode is used, the electrode can be used as an anode or cathode and carry anodic or cathodic current. In some instances, an electrode might be an anode for a period of time and a cathode for a period of time. These and possibly other stimulation parameters taken together comprise a stimulation program that the stimulation circuitry 224 in the IPG 202 can execute to provide therapeutic stimulation to a patient.

In some examples, a measurement device coupled to the muscles or other tissue stimulated by the target neurons, or a unit responsive to the patient or clinician, can be coupled to the IPG 202 or microdrive motor system. The measurement device, user, or clinician can indicate a response by the target muscles or other tissue to the stimulation or recording electrode(s) to further identify the target neurons and facilitate positioning of the stimulating electrode(s). For example, if the target neurons are directed to a muscle experiencing tremors, a measurement device can be used to observe the muscle and indicate changes in, for example, tremor frequency or amplitude in response to stimulation of neurons. Alternatively, the patient or clinician can observe the muscle and provide feedback.

FIGS. 3A-3B illustrate, by way of example and not limitation, leads that may be coupled to the IPG to deliver electrostimulation such as DBS. FIG. 3A shows a lead 301A with electrodes 316A disposed at least partially about a circumference of the lead 301A. The electrodes 316A may be located along a distal end portion of the lead. As illustrated herein, the electrodes 316A are ring electrodes that span 360 degrees about a circumference of the lead 301. A ring electrode allows current to project equally in every direction from the position of the electrode, and typically does not enable stimulus current to be directed from only a particular angular position or a limited angular range around of the lead. A lead which includes only ring electrodes may be referred to as a non-directional lead.

FIG. 3B shows a lead 301B with electrodes 316B including ring electrodes such as E1 at a proximal end and E8 at the distal end. Additionally, the lead 301 also include a plurality of segmented electrodes (also known as split-ring electrodes). For example, a set of segmented electrodes E2, E3, and E4 are around the circumference at a longitudinal position, each spanning less than 360 degrees around the lead axis. In an example, each of electrodes E2, E3, and E4 spans 90 degrees, with each being separated from the others by gaps of 30 degrees. Another set of segmented electrodes E5, E6, and E7 are located around the circumference at another longitudinal position different from the segmented electrodes E2, E3 and E4. Segmented electrodes such as E2-E7 can direct stimulus current to a selected angular range around the lead.

Segmented electrodes can typically provide superior current steering than ring electrodes because target structures in DBS or other stimulation are not typically symmetric about the axis of the distal electrode array. Instead, a target may be located on one side of a plane running through the axis of the lead. Through the use of a radially segmented electrode array, current steering can be performed not only along a length of the lead but also around a circumference of the lead. This provides precise three-dimensional targeting and delivery of the current stimulus to neural target tissue, while potentially avoiding stimulation of other tissue. In some examples, segmented electrodes can be together with ring electrodes. A lead which includes at least one or more segmented electrodes may be referred to as a directional lead. In an example, all electrodes on a directional lead can be segmented electrodes. In another example, there can be different numbers of segmented electrodes at different longitudinal positions.

Segmented electrodes may be grouped into rows of segmented electrodes, where each set is disposed around a circumference at a particular longitudinal location of the directional lead. The directional lead may have any number of segmented electrodes in a given set of segmented electrodes. By way of example and not limitation, a given set may include any number between two to sixteen segmented electrodes. In an example, all rows of segmented electrodes may contain the same number of segmented electrodes. In another example, one set of the segmented electrodes may include a different number of electrodes than at least one other set of segmented electrodes.

The segmented electrodes may vary in size and shape. In some examples, the segmented electrodes are all of the same size, shape, diameter, width or area or any combination thereof. In some examples, the segmented electrodes of each circumferential set (or even all segmented electrodes disposed on the lead) may be identical in size and shape. The rows of segmented electrodes may be positioned in irregular or regular intervals along a length of the lead 201.

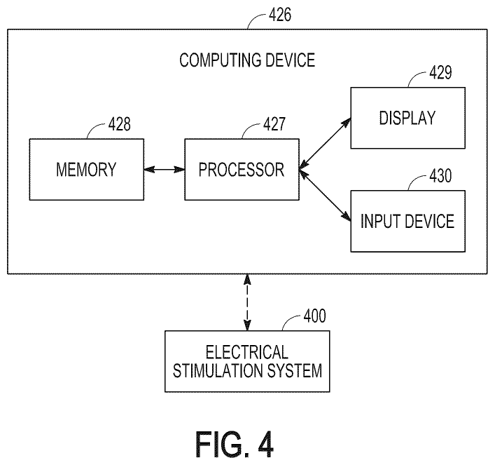

FIG. 4 illustrates, by way of example and not limitation, a computing device 426 for programming or controlling the operation of an electrical stimulation system 400. The computing device 426 may include a processor 427, a memory 428, a display 429, and an input device 430. Optionally, the computing device 426 may be separate from and communicatively coupled to the electrical stimulation system 400, such as system 100 in FIG. 1 Alternatively, the computing device 426 may be integrated with the electrical stimulation system 100, such as part of the IPG 102, RC 103, CP 104, or ETM 105 illustrated in FIG. 1. The computing device may be used to perform process(s) for sensing parameter(s).

The computing device 426, also referred to as a programming device, can be a computer, tablet, mobile device, or any other suitable device for processing information. The computing device 426 can be local to the user or can include components that are non-local to the computer including one or both of the processor 427 or memory 428 (or portions thereof). For example, the user may operate a terminal that is connected to a non-local processor or memory. The functions associated with the computing device 426 may be distributed among two or more devices, such that there may be two or more memory devices performing memory functions, two or more processors performing processing functions, two or more displays performing display functions, and/or two or more input devices performing input functions. In some examples, the computing device 406 can include a watch, wristband, smartphone, or the like. Such computing devices can wirelessly communicate with the other components of the electrical stimulation system, such as the CP 104, RC 103, ETM 105, or IPG 102 illustrated in FIG. 1. The computing device 426 may be used for gathering patient information, such as general activity level or present queries or tests to the patient to identify or score pain, depression, stimulation effects or side effects, cognitive ability, or the like. In some examples, the computing device 426 may prompt the patient to take a periodic test (for example, every day) for cognitive ability to monitor, for example, Alzheimer's disease. In some examples, the computing device 426 may detect, or otherwise receive as input, patient clinical responses to electrostimulation such as DBS, and determine or update stimulation parameters using a closed-loop algorithm based on the patient clinical responses. Examples of the patient clinical responses may include physiological signals (e.g., heart rate) or motor parameters (e.g., tremor, rigidity, bradykinesia). The computing device 426 may communicate with the CP 104, RC 103, ETM 105, or IPG 102 and direct the changes to the stimulation parameters to one or more of those devices. In some examples, the computing device 426 can be a wearable device used by the patient only during programming sessions. Alternatively, the computing device 426 can be worn all the time and continually or periodically adjust the stimulation parameters. In an example, a closed-loop algorithm for determining or updating stimulation parameters can be implemented in a mobile device, such as a smartphone, which is connected to the IPG or an evaluating device (e.g., a wristband or watch). These devices can also record and send information to the clinician.

The processor 427 may include one or more processors that may be local to the user or non-local to the user or other components of the computing device 426. A stimulation setting (e.g., parameter set) includes an electrode configuration and values for one or more stimulation parameters. The electrode configuration may include information about electrodes (ring electrodes and/or segmented electrodes) selected to be active for delivering stimulation (ON) or inactive (OFF), polarity of the selected electrodes, electrode locations (e.g., longitudinal positions of ring electrodes along the length of a non-directional lead, or longitudinal positions and angular positions of segmented electrodes on a circumference at a longitudinal position of a directional lead), stimulation modes such as monopolar pacing or bipolar pacing, etc. The stimulation parameters may include, for example, current amplitude values, current fractionalization across electrodes, stimulation frequency, stimulation pulse width, etc.

The processor 427 may identify or modify a stimulation setting through an optimization process until a search criterion is satisfied, such as until an optimal, desired, or acceptable patient clinical response is achieved. Electrostimulation programmed with a setting may be delivered to the patient, clinical effects (including therapeutic effects and/or side effects, or motor symptoms such as bradykinesia, tremor, or rigidity) may be detected, and a clinical response may be evaluated based on the detected clinical effects. When actual electrostimulation is administered, the settings may be referred to as tested settings, and the clinical responses may be referred to as tested clinical responses. In contrast, for a setting in which no electrostimulation is delivered to the patient, clinical effects may be predicted using a computational model based at least on the clinical effects detected from the tested settings, and a clinical response may be estimated using the predicted clinical effects. When no electrostimulation is delivered the settings may be referred to as predicted or estimated settings, and the clinical responses may be referred to as predicted or estimated clinical responses.

In various examples, portions of the functions of the processor 427 may be implemented as a part of a microprocessor circuit. The microprocessor circuit can be a dedicated processor such as a digital signal processor, application specific integrated circuit (ASIC), microprocessor, or other type of processor for processing information. Alternatively, the microprocessor circuit can be a processor that can receive and execute a set of instructions of performing the functions, methods, or techniques described herein.

The memory 428 can store instructions executable by the processor 427 to perform various functions including, for example, determining a reduced or restricted electrode configuration and parameter search space (also referred to as a “restricted search space”), creating or modifying one or more stimulation settings within the restricted search space, etc. The memory 428 may store the search space, the stimulation settings including the “tested” stimulation settings and the “predicted” or “estimated” stimulation settings, clinical effects (e.g., therapeutic effects and/or side effects) and clinical responses for the settings.

The memory 428 may be a computer-readable storage media that includes, for example, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information, and which can be accessed by a computing device.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, Bluetooth, near field communication, and other wireless media.

The display 429 may be any suitable display or presentation device, such as a monitor, screen, display, or the like, and can include a printer. The display 429 may be a part of a user interface configured to display information about stimulation settings (e.g., electrode configurations and stimulation parameter values and value ranges) and user control elements for programming a stimulation setting into an IPG. The computing device 426 may include other output(s) such as speaker(s) and haptic output(s) (e.g., vibration motor).

The input device 430 may be, for example, a keyboard, mouse, touch screen, track ball, joystick, voice recognition system, or any combination thereof, or the like. Another input device 430 may be a camera from which the clinician can observe the patient. Yet another input device 430 may a microphone where the patient or clinician can provide responses or queries.

The electrical stimulation system 400 may include, for example, any of the components illustrated in FIG. 1. The electrical stimulation system 400 may communicate with the computing device 426 through a wired or wireless connection or, alternatively or additionally, a user can provide information between the electrical stimulation system 400 and the computing device 426 using a computer-readable medium or by some other mechanism.

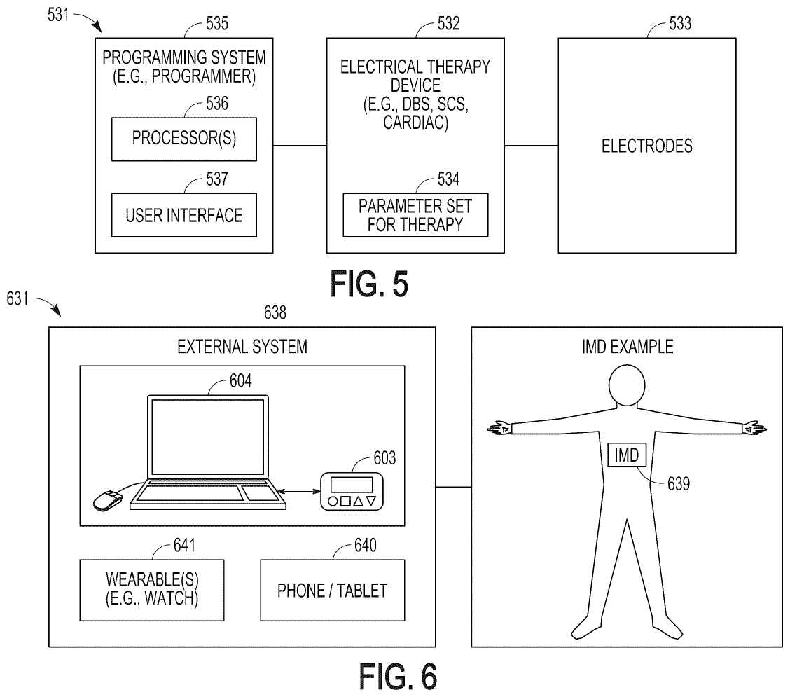

FIG. 5 illustrates, by way of example, an example of an electrical therapy-delivery system. The illustrated system 531 includes an electrical therapy device 532 configured to deliver an electrical therapy to electrodes 533 to treat a condition in accordance with a programmed parameter set 534 for the therapy. The system 531 may include a programming system 535, which may function as at least a portion of a processing system, which may include one or more processors 536 and a user interface 537. The programming system 535 may be used to program and/or evaluate the parameter set(s) used to deliver the therapy. The illustrated system 531 may be a DBS system.

In some embodiments, the illustrated system 531 may include an SCS system to treat pain and/or a system for monitoring pain. By way of example, a therapeutic goal for conventional SCS programming may be to maximize stimulation (i.e., recruitment) of the dorsal column (DC) fibers that run in the white matter along the longitudinal axis of the spinal cord and minimal stimulation of other fibers that run perpendicular to the longitudinal axis of the spinal cord (e.g., dorsal root fibers).

A therapy may be delivered according to a parameter set. The parameter set may be programmed into the device to deliver the specific therapy using specific values for a plurality of therapy parameters. For example, the therapy parameters that control the therapy may include pulse amplitude, pulse frequency, pulse width, and electrode configuration (e.g., selected electrodes, polarity and fractionalization). The parameter set includes specific values for the therapy parameters. The number of electrodes available combined with the ability to generate a variety of complex electrical waveforms (e.g., pulses), presents a huge selection of modulation parameter sets to the clinician or patient. For example, if the neuromodulation system to be programmed has sixteen electrodes, millions of modulation parameter sets may be available for programming into the neuromodulation system. To facilitate such selection, the clinician generally programs the modulation parameters sets through a computerized programming system to allow the optimum modulation parameters to be determined based on patient feedback or other means and to subsequently program the desired modulation parameter sets.

FIG. 6 illustrates, by way of example and not limitation, a monitoring system and/or the electrical therapy-delivery system of FIG. 5, implemented using an implantable medical device (IMD). The illustrated system 631 includes an external system 638 that may include at least one programming device. The illustrated external system 638 may include a clinician programmer 604, similar to CP 104 in FIG. 1, configured for use by a clinician to communicate with and program the neuromodulator, and a remote control device 603, similar to RC 103 in FIG. 1, configured for use by the patient to communicate with and program the neuromodulator. For example, the remote control device 603 may allow the patient to turn a therapy on and off, change or select programs, and/or may allow the patient to adjust patient-programmable parameter(s) of the plurality of modulation parameters. FIG. 6 illustrates an IMD 639, although the monitor and/or therapy device may be an external device such as a wearable device. The external system 638 may include a network of computers, including computer(s) remotely located from the IMD 639 that are capable of communicating via one or more communication networks with the programmer 604 and/or the remote control device 603. The remotely located computer(s) and the IMD 639 may be configured to communicate with each other via another external device such as the programmer 604 or the remote control device 603. The remote control device 603 and/or the programmer 604 may allow a user (e.g., patient and/or clinician or rep) to answer questions as part of a data collection process. The external system 638 may include personal devices such as a phone or tablet 640, wearables such as a watch 641, sensors or therapy-applying devices. The watch may include sensor(s), such as sensor(s) for detecting activity, motion and/or posture. Other wearable sensor(s) may be configured for use to detect activity, motion and/or posture of the patient. The external system 638 may include, but is not limited to, a phone and/or a tablet. Notifications may be sent to the patient, physician, device rep or other users via the external system and through remote portals (e.g., web-based portals) provided by remote systems.

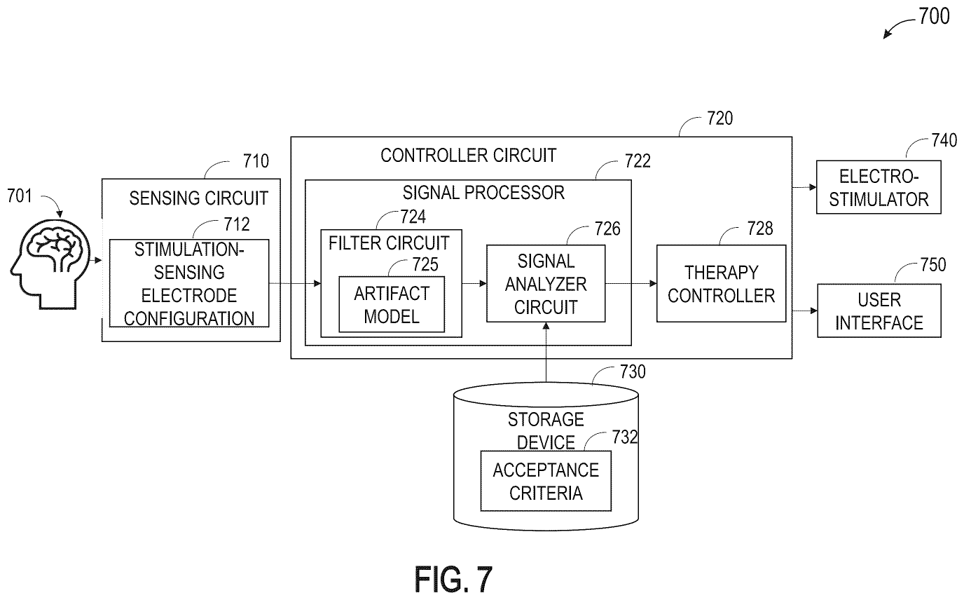

FIG. 7 illustrates an example of a neuromodulation system 700 configured to provide guided implantation (e.g., lead placement) and device programming based on selected evoked responses. The system 700 includes a sensing circuit 710, a controller circuit 720, a storage device 730, an electrostimulator 740, and a user interface 750. Portions of the system 700 may be implemented in the IPG 102 or the CP 104.

The sensing circuit 710 may be operatively connected to one or more leads and electrodes associated therewith, such as ring electrodes or segmented electrodes on the non-directional lead 301A or the directional lead 301B. The ring electrodes and/or the segmented electrodes may also be electrically coupled to the electrostimulator 740. The ring electrodes and/or the segmented electrodes may be configured as sensing electrodes for sensing ERs, or as stimulating electrodes for delivering electrostimulation pulses. The sensing circuit 710 may sense ERs from one or more sensing electrodes on a lead placed at target issue (e.g., STN) of a patient 701 in response to electrostimulation pulses delivered from a stimulating electrode at a stimulation site (e.g., a brain target).

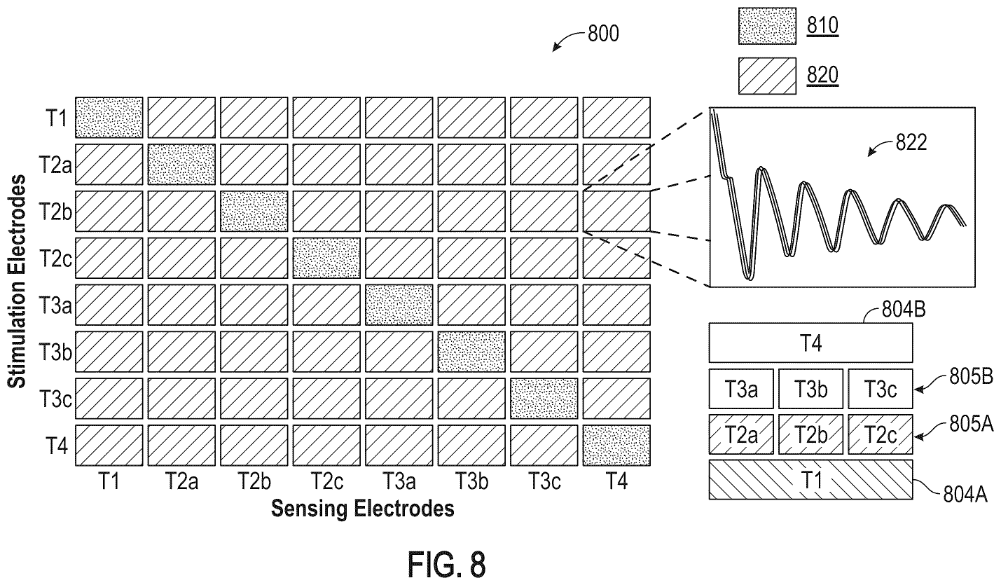

The ERs may be sensed in accordance with a stimulating-sensing electrode configuration 712. FIG. 8 illustrates an example of the stimulating-sensing electrode configuration 712, graphically represented by an ER sensing map 800 including a two-dimensional (2D) array where the sensing electrodes are indexed on the horizontal axis, and the stimulating electrodes are indexed on the vertical axis. The sensing electrodes and the stimulating electrodes are each selected from electrodes on a portion of a DBS lead. By way of example and not limitation, the electrodes include ring electrodes 804A (electrode “T1”) and 804B (electrode “T4”) and two rows of segmented electrodes 805A and 805B, all arranged in-line along the DBS lead. The two rows of segmented electrodes 805A and 805B each include three segmented electrodes (T2a, T2b, and T2c in 805A, T3a, T3b, and T3c in 805B) arranged about a circumference of the DBS lead. Other number or arrangement of electrodes (ring electrodes and/or segmented electrodes) may be included along the lead. In some examples, the sensing electrodes or the stimulating electrodes may be selected from electrodes not on the DBS leads. For example, at least some sensing electrodes or the stimulating electrodes may be selected from skin patch electrodes. Other nomenclatures and methods of describing the evoking and recording electrodes may be used, including those which involve multiple evoking electrodes with proportioned or fractionalized current, or Multiple Independent Current Control (MICC) to generate precise control to refine the size and shape of the stimulation field, designed to customize therapy for individual patients.

The diagonal elements in the 2D array as shown in the ER sensing map 800 represent “on-diagonal” sensing configuration where the same electrode is used for delivering electrostimulation pulses and for recording an ER to that electrostimulation in the same stimulation session. Such electrode is also referred to as “on-diagonal” sensing electrode, and the ER sensed therefrom is referred to as “on-diagonal” ER 810. The off-diagonal elements in the 2D array represent an “off-diagonal” sensing configuration where different electrodes are used for delivering electrostimulation pulses and for recording an ER to that electrostimulation in the same stimulation session. The sensing electrodes are also referred to as “off-diagonal” sensing electrodes, and the ER sensed therefrom is referred to as “off-diagonal” ER 820. The ERs may be recorded in multiple stimulation-ER recording sessions. For example, when electrostimulation pulses are delivered from electrode T1, an “on-diagonal” ER may be recorded from electrode T1, and “off-diagonal” ERs may be recorded from one or more of the rest of the electrodes (T2a-Tac, Ta3a-T3c, and T4). The stimulation-ER recording session can be repeated when stimulation is delivered from other electrodes. As an example, off-diagonal ERs 822 are recorded from sensing electrode T4 in response to stimulation delivered at electrode T2b. ERs recorded in accordance with the ER sensing map 800 can then be analyzed such as to determine a spatial distribution of ERs, which may then be used to guide lead placement and device programming, as will be discussed further below.

Recording ERs in accordance with the full ER sensing map (i.e., to include both the “on-diagonal” and “off-diagonal” configurations) as depicted in FIG. 8 can be time consuming and take up a large amount of system sources and memory spaces. Additionally, Although the “on-diagonal” ERs at the stimulation site may provide useful information about ER distribution and therefore for identifying a match to the desired or target response, the “on-diagonal” ERs are prone to stimulation artifacts strong enough to contaminate the ER component of interest. Inclusion of the “on-diagonal” ERs in ER analysis such as estimation of spatial distribution may introduce errors and deteriorate the quality of ER-based lead placement and/or device programming. To avoid such effect, in an example, the stimulating-sensing electrode configuration 712 may be defined to include only the “off-diagonal” electrodes but not the “on-diagonal” electrodes, and only the “off-diagonal” ERs are used for determining a spatial distribution of ERs and for guiding the lead placement and device programming.

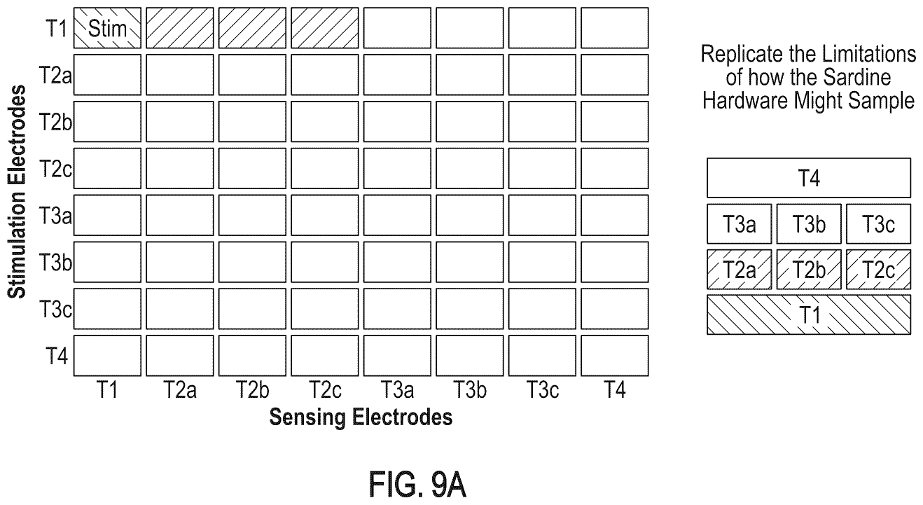

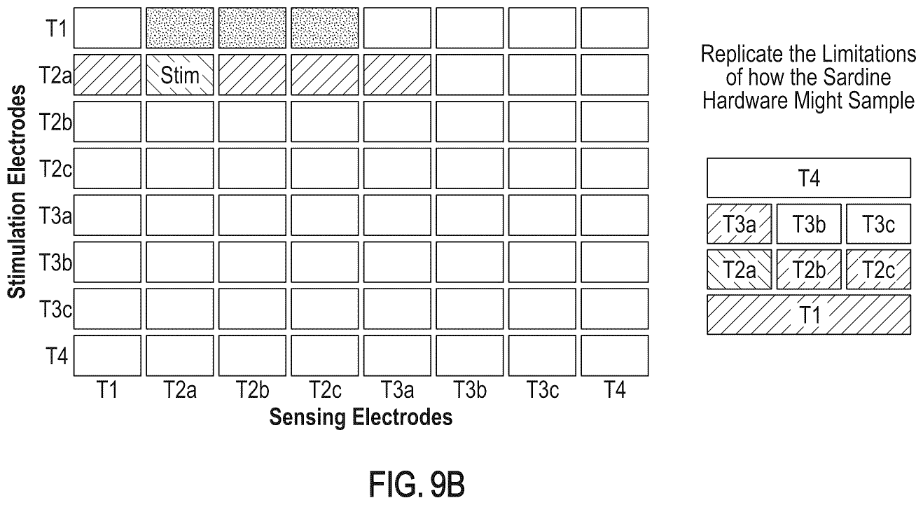

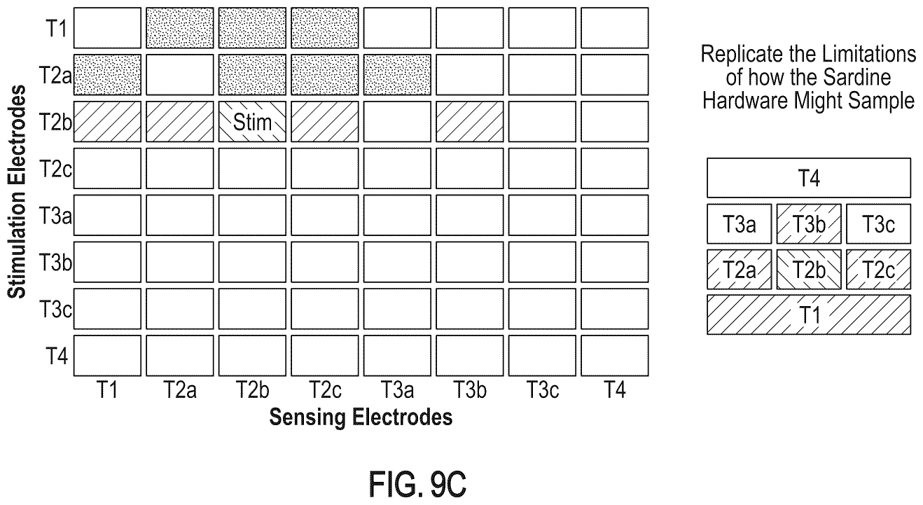

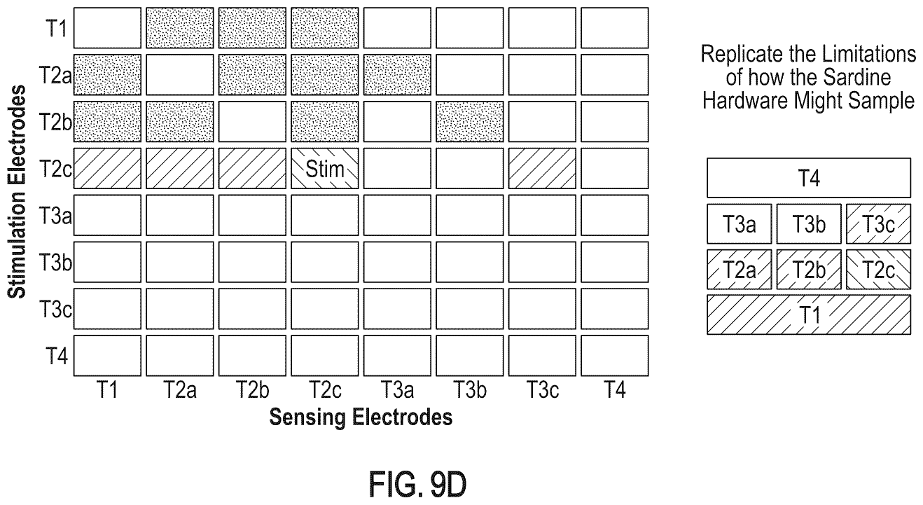

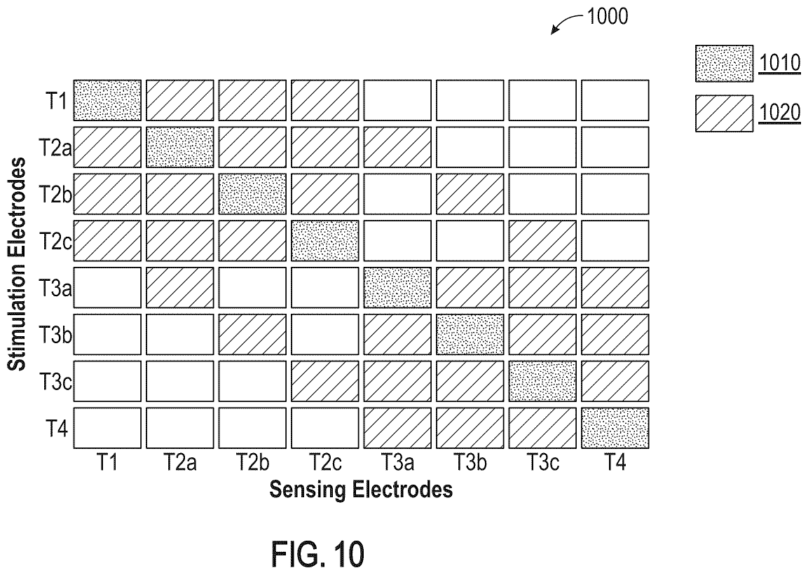

As an alternative to the “off-diagonal” sensing configuration that employs all available electrodes except the stimulating electrode for ER sensing, FIGS. 9A-9D and FIG. 10 illustrate an example of a stimulating-sensing electrode configuration that involves a selected subset, less than an entirety, of available electrodes for ER sensing. The selected subset can include non-diagonal electrodes within a specific proximity or with a specific geometric relationship to the stimulating electrode being used for delivering electrostimulation pulses. One such ER sensing configuration is also referred to as a “nearest neighbor” configuration. In an example, the nearest neighbor configuration includes two or more sensing electrodes immediate adjacent to the stimulating electrode on the lead. By way of example and not limitation, FIG. 9A shows, in response to electrostimulation delivered at stimulating electrode T1, ERs sensed only from three nearest neighbor electrodes T2a, T2b, and T2c of the row of segmented electrodes 805A. FIG. 9B shows, in response to electrostimulation delivered at stimulating electrode T2a, ERs sensed only from four nearest neighbor electrodes including T2b and T2c of the row of segmented electrodes 805A, T3a of the row of segmented electrodes 805B, and the ring electrode T1. FIG. 9C shows, in response to electrostimulation delivered at stimulating electrode T2b, ERs sensed only from four nearest neighbor electrodes including T2a and T2c of the row of segmented electrodes 805A, T3b of the row of segmented electrodes 805B, and the ring electrode T1. FIG. 9D shows, in response to electrostimulation delivered at stimulating electrode T2c, ERs sensed only from four nearest neighbor electrodes including T2a and T2b of the row of segmented electrodes 805A, T3c of the row of segmented electrodes 805B, and the ring electrode T1. The above stimulation-ER recording sessions continue with stimulating at T3a, T3b, T3c, and T4 electrodes and recording at respective three or four nearest neighbor “non-diagonal” electrodes. FIG. 10 illustrates an ER sensing map 1000 where the ERs 1020 are sensed from the “nearest neighbor” electrodes with respect to each stimulating electrode as depicted in FIGS. 9A-9D. Similar to the ER sensing map 800, the “on-diagonal” ERs 1010 may be excluded from the ERs being used for estimating a spatial distribution of ERs and for guiding the lead placement and device programming.

The controller circuit 720 can include circuit sets comprising one or more other circuits or sub-circuits, such as a signal processor 722 and a therapy controller 728. The signal processor 722 may further include a filter circuit 724 and a signal analyzer circuit 726. The circuits or sub-circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

In various examples, portions of the functions of the controller circuit 720 may be implemented as a part of a microprocessor circuit. The microprocessor circuit can be a dedicated processor such as a digital signal processor, application specific integrated circuit (ASIC), microprocessor, or other type of processor for processing information including physical activity information. Alternatively, the microprocessor circuit can be a general purpose processor that can receive and execute a set of instructions of performing the methods or techniques described herein.

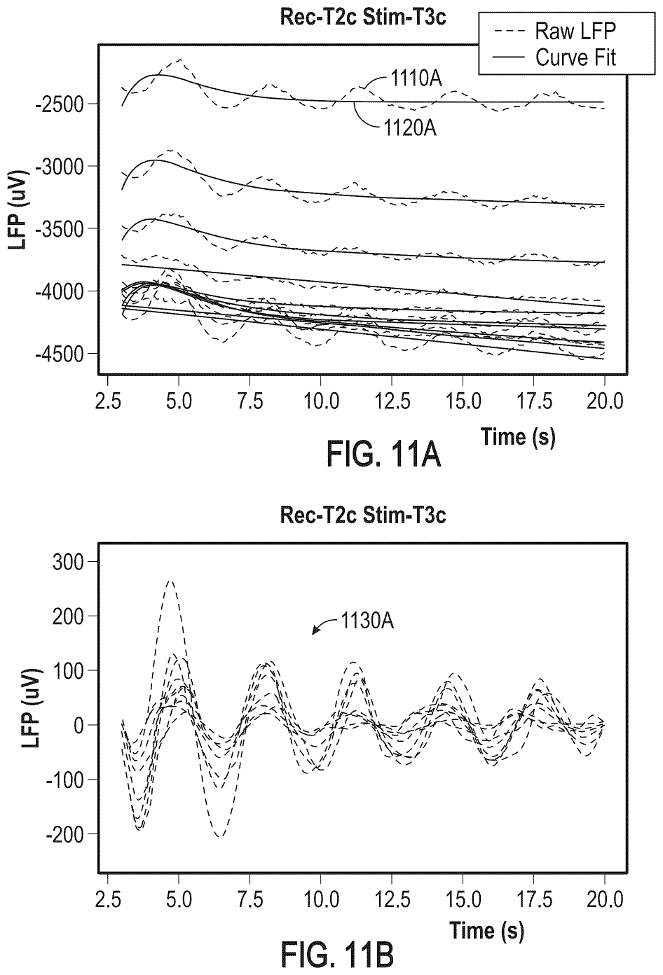

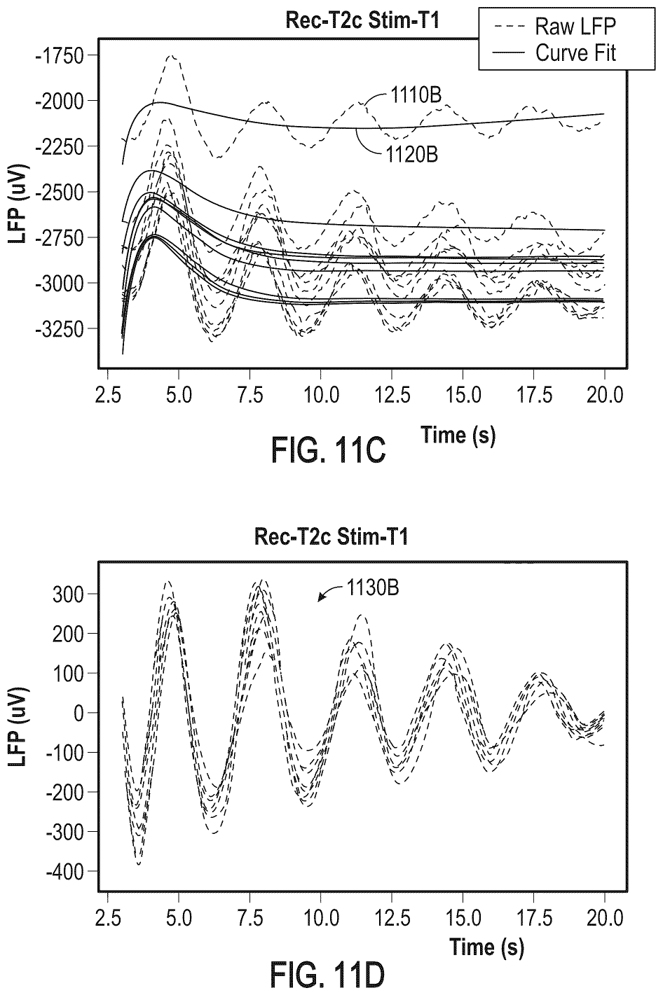

The filter circuit 724 can include a filter or a filter bank to filter the recorded ER signals. As discussed above, although the “on-diagonal” ERs 810 generally provide useful information about the distribution of ERs, they are prone to stimulation artifacts which may contaminate the tissue ER component of interest. Additionally, stimulation and other artifacts may also present in some “off-diagonal” ERs particularly those from sensing electrodes in close proximity of the stimulating electrode, such as the group of “nearest neighbor” electrodes as discussed above with respect to FIGS. 9A-9D and 10. By way of example and not limitation, FIGS. 11A-11D illustrate example effects of filtering the ERs sensed from nearest neighbor electrodes (such as in accordance with the ER sensing map 1000) to remove or substantially attenuate stimulation and other artifacts modeled by a polynomial-exponential decay model. The ER recordings can be obtained in multiple stimulation-ER recording sessions during which stimulation pulses are delivered via a stimulating electrode with varying stimulation parameter settings (e.g., stimulation amplitude, frequency, or pulse width). FIG. 11A illustrates raw ER signals 1110A from sensing electrode T2c in response to electrostimulation delivered via stimulating electrode T3c at varying stimulation parameter values. FIG. 11C illustrates raw ER signals 1110B from sensing electrode T2c in response to electrostimulation delivered via stimulating electrode T1 at varying stimulation parameter values.

The ER recordings from each of the nearest neighbor sensing electrode contains a stimulation artifact component in the form of a decay trend, and an oscillatory component representing tissue ER of interest. Such ER recordings are also referred to as decaying-oscillation evoked potentials. The stimulation artifact, if not removed or sufficiently suppressed, may introduce error in ER signal analysis (e.g., ER feature and ER distribution) and reduce the reliability of finding a match to the desired or target evoked response, thereby leading to suboptimal lead placement and/or programming of the electrostimulator 740. Such stimulation artifacts are conventionally managed using hardware components and techniques such as analog filters and digital-to-analog converters (DACs) with sufficiently wide active range and enough steps. The hardware, however, can be large and the settings are usually inconvenient to adjust. The present document describes a model-based artifact characterization and removal technique to improve signal quality of ERs. As illustrated in FIG. 7, the filter circuit 724 may model the artifact component of the ER signal using an artifact model 725. In an example, the artifact model can be a parametric model. The artifact model 725 can be trained to fit the artifact component using a least-mean squared (LMS) method, or other methods to minimize a fitting error. Examples of the parametric model include an exponential model, a polynomial model, a linear combination (e.g., sum or weighted sum) of exponentials model, or a nonlinear combination of exponentials model. In an example, the parametric model is a polynomial-exponential decay model that includes both exponential terms and polynomial terms, as given in Equation (1) below:

$$f ( t ) = c + {a}_{o} {e}^{{b}_{o} t} + {a}_{1} {te}^{{b}_{1} t} \qquad ( 1 )$$

In Equation (1), a₀, a₁, b₀, b₁, and c are model parameters (constants) that can be determined using LMS method or other method. The parameter model according to Equation (1) can be depicted as a fitted curve. The filter circuit 724 can remove or substantially attenuate the stimulation artifact by subtracting the fitted curve from the raw ER signal. The residual signals after subtraction are oscillatory components representing tissue ER of interest. For example, FIG. 11A shows a fitted curve 1120A that models the stimulation artifact component in the raw ER signal 1110A using the polynomial-exponential decay model. FIG. 11B shows residual signals 1130A after subtracting the fitted curve 1120A from the raw ER signal 1110A. FIG. 11C shows the a fitted curve 1120B that models the stimulation artifact component in the raw ER signal 1110B using the polynomial-exponential decay model. FIG. 11D shows residual signals 1130B after subtracting the fitted curve 1120B from the raw ER signal 1110B. The filter circuit 724 may further pass the residual signal to a bandpass filter or a lowpass filter to remove or substantially attenuate noise or other sources of interferences.

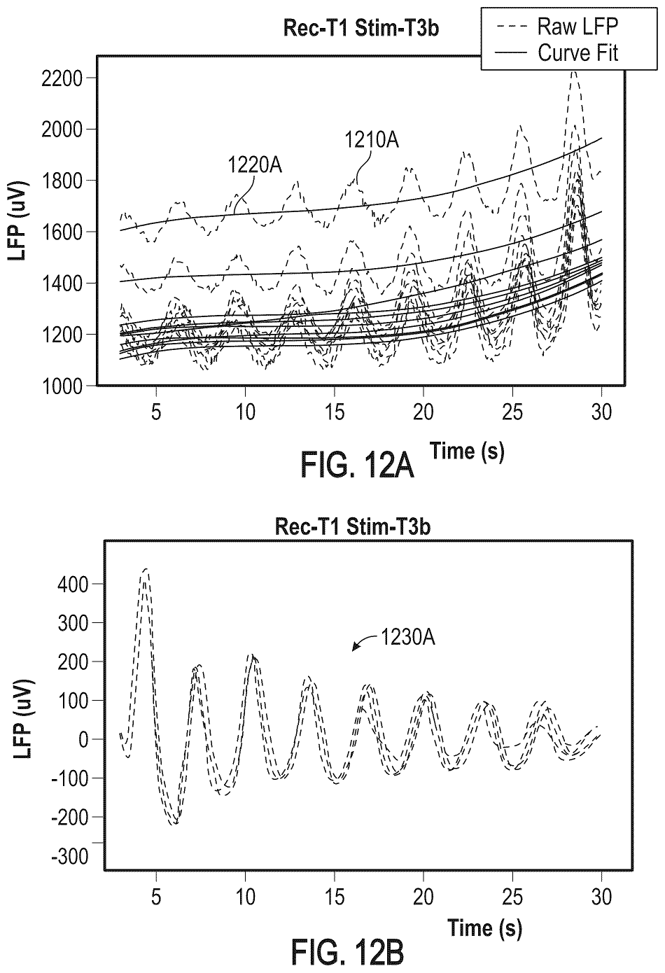

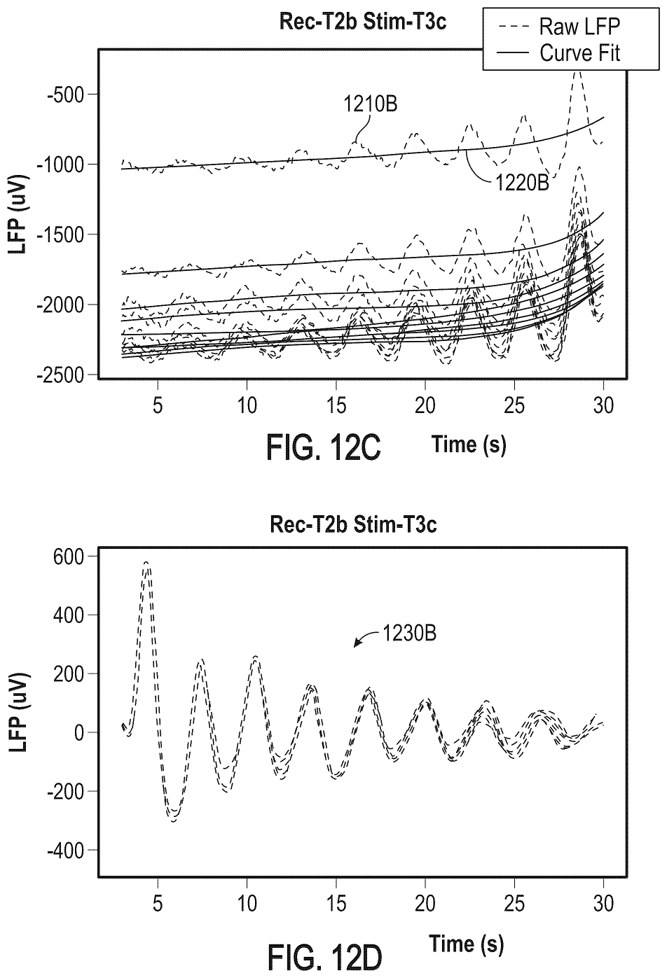

In certain cases, curve fitting using an exponential model or a variant thereof such as the polynomial-exponential model as shown in Equation (1) may introduce overfitting. Overfitting may reduce the model's generalization capability and produce less accurate or less robust modelling of stimulation artifact. In the examples illustrated in FIGS. 11A and 11C, overfitting is more likely to occur at the beginning of the ER recording (i.e., immediately after electrostimulation) than at the end of the ER recording. To prevent or reduce overfitting, in an example, the filter circuit 724 may generate a time-reversed version of the raw ER signal, and apply the polynomial-exponential decay model (or other models) to the time-reversed ER signal to remove or substantially attenuate the artifact component. By way of example, FIGS. 12A-12D illustrate example effects of filtering time-reversed version of ERs sensed from nearest neighbor electrodes to remove or substantially attenuate stimulation artifacts modeled by a polynomial-exponential decay model. Specifically, FIG. 12A illustrates time-reversed ER signals 1210A from sensing electrode T1 in response to electrostimulation delivered via stimulating electrode T3b at varying stimulation parameter values (e.g., stimulation amplitude, frequency, or pulse width), and a fitted curve 1220A that models the stimulation artifact component in the time-reversed ER signal 1210A. FIG. 12B shows residual signals 1230A after subtracting the fitted curve 1220A from the time-reversed ER signals 1210A. FIG. 12C illustrates time-reversed ER signals 1210B from sensing electrode T2b in response to electrostimulation delivered via stimulating electrode T3c at varying stimulation parameter values, and a fitted curve 1220B that models the stimulation artifact component in the time-reversed ER signal 1210B. FIG. 12D shows residual signals 1230B after subtracting the fitted curve 1220B from the time-reversed ER signal 1210B. As shown in FIGS. 12B and 12D, time-reversing the ER signal can advantageously delay the potential overfitting of stimulation artifact component to the end of the time-reversed ER signal. The degree of overfitting in the time-reversed ER can also be significantly reduced.

In some examples, the electrostimulator 740 may intermittently pause the delivery of electrostimulation pulses. During the intermittent pause, the sensing circuit 710 can collect the ERs from the nearest neighbor sensing electrodes (such as in accordance with FIG. 10). As the stimulation artifacts are more likely to occur at high stimulation amplitude, high pulse width, or high stimulation frequency (pulse rate), introducing the intermittent pause can effectively prevent occurrence or reduce the degree of stimulation artifacts in ER recordings. In an example, the controller circuit 720 can control the electrostimulator 740 to initiate, or adjust an amount of, intermittent pause based on the stimulation setting, such as when one or more stimulation amplitude, pulse width, or stimulation frequency exceed respective thresholds. The pauses can vary, and be unequal in duration. The variation in the pauses may be used to optimize the saturation or stimulation artifact.

The signal analyzer circuit 726 may extract a signal feature from the filtered ER signal (e.g., the ER signal with the stimulation artifact removed). Examples of the signal features can include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, can be measured as a difference between a maximum value or a minimum value of a dominant peak in the sensed evoked response or an epoch-averaged evoked response within the time window (also referred to as “max P2P” amplitude). Alternatively, the P2P value may be measured as a difference between a negative peak (trough) and an immediate subsequent positive peak (also referred to as “N1-P2 P2P” amplitude). The signal curve length can be measured as accumulated signal value differences of the sensed evoked response (or an epoch-averaged evoked response) over consecutive unit times (e.g., consecutive data sampling intervals) within the time window. The signal power can be measured as an area under the curve (AUC) of the sensed evoked response (or the epoch-averaged evoked response) within the time window. In some examples, the signal analyzer circuit 726 may generate a spatial distribution of extracted signal features across sensing locations of the sensing electrodes, such as the selected group of sensing electrodes as illustrated in FIG. 10.

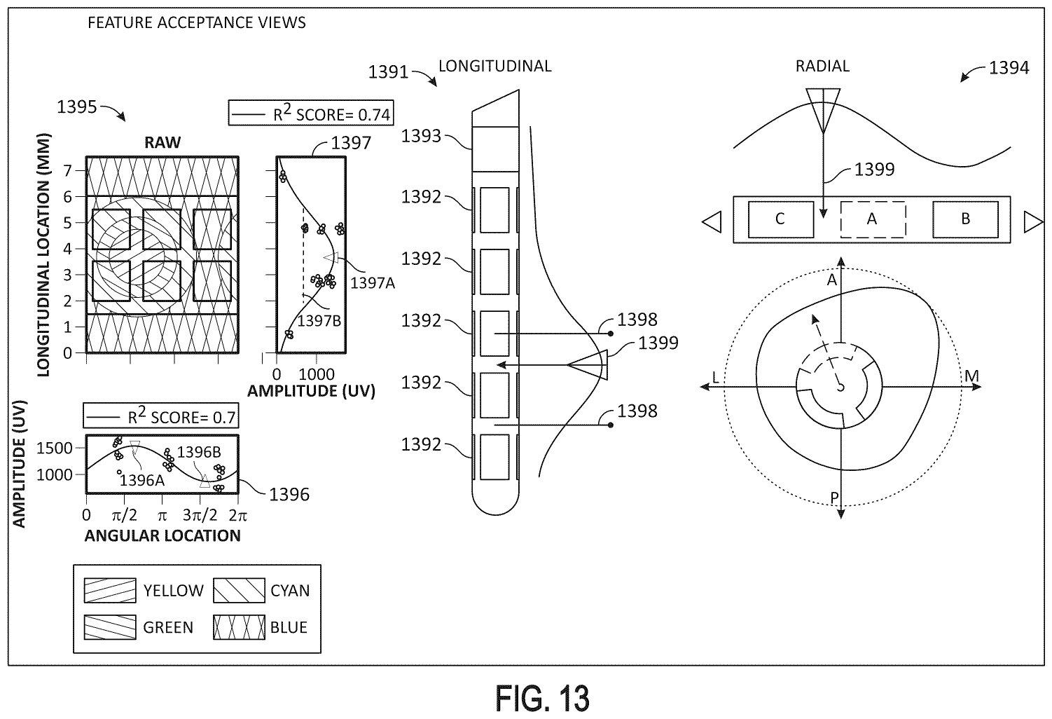

The signal analyzer circuit 726 may compare the filtered ERs, or signal features or a spatial distribution of signal features derived therefrom, to one or more acceptance criteria 732 to determine whether a match to the desired or target response can be found. In some examples, the acceptance criteria are set, modulated, inspected, accepted by the clinical user, including ahead of or during operation. In some examples, the signal analyzer circuit 726 may accumulate the sensed ERs obtained in multiple stimulation-ER recording sessions during which stimulation pulses are delivered via a particular stimulating electrode with varying stimulation parameter settings (e.g., stimulation amplitude, frequency, or pulse width), such as those shown in FIGS. 11A, 11C, 12A, and 12C, and compare the accumulated ERs (or signal features or a spatial distribution of signal features derived from the filtered ERs) to the acceptance criteria 732. The acceptance criteria 732 can be provided by a user such as via the user interface 750. Alternatively, the acceptance criteria 732 can be predetermined and stored in a storage device 730 accessible by the signal analyzer circuit 726. In an example, the acceptance criteria 732 is a user-provided acceptance ER signal bounds (e.g., upper and lower bounds, location bounds, properties bounds such presence, absence, or value of a feature). In another example, the acceptance criteria includes a target ER template representing a patient-specific ER or a population-based ER to electrostimulation of the neural target. In an example, the target ER template may be selected to relieve symptoms or for other goals such as lead placement for disease-modifying therapy or co-therapy (e.g., leads that inject drugs or light), and side-effect avoidance. In some examples, the acceptance criteria may include one of a plurality of candidate ER templates indexed by region, clinical institution, group, participants information, implanter information, or symptom relief goals, and a target ER template can be selected from the plurality of candidate ER templates based at least in part on one or more of an identification of an institution where the patient is implanted or treated with the electrostimulation, a group, or the participants information, an identification of an implanter that implants the lead, or the sensed indication of symptom relief of the patient. In some examples, the target ER template includes a target distribution of ER features. The signal analyzer circuit 726 may determine a distribution of sensed ER features, compare the determined distribution of sensed ERs to the target distribution of ER features to determine whether a match can be found. In some examples, the target ER template includes at least one target representative ER feature (such as a feature derived from the distribution of the ER feature). The signal analyzer circuit 726 may determine at least one measured ER feature from the sensed ERs or from the distribution of sensed ER features, and compare the at least one measured ER feature to the at least one target representative ER feature to determine whether a match can be found. Examples of ER signal features, ER templates, and spatial distribution of the ER signal features are discussed below with respect to FIGS. 13-15.

The therapy controller 728 can generate a control signal to the electrostimulator 740 to adjust the neuromodulation therapy based on the selected signal feature. The electrostimulator 740 may be configured to deliver electrical stimulation according to a stimulation setting. The electrical stimulation may be delivered using a monopolar (far-field) or a bipolar (near-field) configuration. Examples of the therapy setting may include, electrode selection and configuration, stimulation parameter values including, for example, amplitudes, pulse width, frequency, pulse waveform, active or passive recharge mode, ON time, OFF time, therapy duration, and fractionalization, among others. In an example, the therapy controller 728 can be implemented as a proportional integral (PI) controller, a proportional-integral-derivative (PID) controller, or other suitable controller that takes the comparison of the sense ERs (or features or a distribution of the features thereof) to the acceptance criteria 752 as a feedback on the adjustment of stimulation settings. The types of data, and the recordings used to produce them, may vary regarding the type of acceptance criteria and operations employed. For example, ER data used to drive decisions about the electrode selection and configuration may differ from data and evoke/record configurations used to compare to acceptance criteria and use as control signal for amplitude adjustment. One ER measurement may be used to inform lead positioning (e.g., by sweeping a non-therapeutic sampling pulse across the space of the lead electrodes), another ER measurement may be used to determine or adjust a stimulation parameter (e.g., by sweeping a therapeutic sampling pulse across amplitudes).

The electrostimulator 740 can be an implantable module, such as incorporated within the IPG 10. Alternatively, the electrostimulator 740 can be an external stimulation device, such as incorporated with the ETS 40. In some examples, the user can choose to either send a notification (e.g., to the RC 45 or a smartphone with the patient) for a therapy reminder, or to automatically initiate or adjust neuromodulation therapy in accordance with the adjusted therapy setting. If an automatic therapy initiation is selected, the electrostimulator 740 can deliver stimulation in accordance with the adjusted therapy setting.

In some examples, the therapy controller 728 can generate a recommendation to the user to reposition the lead or to adjust the device setting (e.g., a programmable parameter of the electrostimulator 740) to cause the sensed ERs to align with or to compare more favorably to one or more acceptance criteria (e.g., an ER template) during an implantation procedure. In some embodiments the display may provide a suggestion to the user to adjust stimulation parameters to cause the since developed responses to more favorably compared to the one or more acceptance criteria (e.g., an ER template). The recommendation can be displayed on the user interface 750. The user interface 750 can be a portable (e.g., handheld) device, such as the RC 45 or a smartphone (with executable software application) operable by the patient at their home without requiring extra clinic visits or consultation with a device expert. In another example, the user interface 750 can be a programmer device, such as the CP 50. In addition to the recommendation for lead replacement, other information may be displayed on the user interface 750 including, by way of example and not limitation, one or more of the sensed ERs (including, for example, before and/or after artifact removal as illustrated in FIGS. 11-12), ER features, distribution of ER features, the acceptance criteria (e.g., one or more ER templates), or the comparison between the sensed ERs and the acceptance criteria. In some examples, the therapy controller 728 may determine or modify therapeutic stimulation settings based on the sense ERs or features thereof. The electrostimulator 740 may deliver therapeutic stimulation (e.g., DBS) in accordance with the determined or modified therapeutic stimulation settings.

In some examples, the user interface 750 allows a physician to remotely review therapy settings and treatment history, consult with the patient to obtain information including pain relief and SCS-related side effects or symptoms, perform remote programming of the electrostimulator 740, or provide other treatment options to the patient. The user interface 750 can allow a user (e.g., the patient, the physician managing the patient, or a device expert) to view, program, or modify a device setting. For example, the user may use one or more user interface (UI) control elements to provide or adjust values of one or more device parameters, or select from a plurality of pre-defined stimulation programs for future use. Each stimulation program can include a set of stimulation parameters with respective pre-determined values. In some examples, the user interface 750 can include a display to display textually or graphically information provided by the user via an input unit, and device settings including, for example, feature selection, sensing configurations, signal pre-processing settings, therapy settings, optionally with any intermediate calculations. In an example, the user interface 750 may present to the user an “optimal” or improved therapy setting, such as determined based on a closed-loop or adaptive feedback control of electrostimulation based on a selected evoked response signal feature, in accordance with various embodiments discussed in this document. In some examples, the user can use the user interface 750 to provide feedback on a neuromodulation therapy, including, for example, side effects or symptoms arise or persist associated with the neurostimulation, or severity of the symptom or a side effect.

FIG. 13 illustrates, by way of example and not limitation, a display for user interface that creates a response fit from a distribution of ERs. The figure illustrates a longitudinal view of a portion of a lead 1391 including rows of segmented electrodes 1392 and a ring electrode 1393. The segmented electrodes 1392 are illustrated as three electrode per row (see electrodes a, b and c in the radial view 1394). The present subject matter is not limited to this particular example of a lead with this particular arrangement of the electrodes. Also shown in FIG. 13 is a depiction 1395 of a portion of the lead 1391 that includes two rows of segmented electrodes (each including three segmented electrodes) arranged in-line along the lead 1391 between two ring electrodes, a configuration similarly shown in FIG. 8. Overlaid upon the electrode depiction 1395 includes a hotspot view that provides an indicator (e.g., color heatmap) for a hotspot for the distribution of ERs or ER features, along with a directional depiction 1396 of the distribution of ER features across angular locations of the segmented electrodes on a circumference of the lead, and a longitudinal depiction 1397 of the distribution of ER features across longitudinal locations of electrodes along the lead. In the illustrated example, the ER features include ER magnitude, which can be computed as “max P2P” amplitude or “N1-P2 P2P” amplitude as described above with respect to FIG. 7. Other ER features can be used. By way of example, the directional depiction 1396 shows a wrapped Gaussian distribution of ER features across angular locations of the segmented electrodes on a circumference of the lead. The longitudinal depiction 1397 shows a Gaussian distribution of the ER features across longitudinal locations of electrodes along the lead. One or more model parameters or features may be derived from each of the ER distributions. In one example, the model parameters may include a positive peak amplitude 1396A or a negative peak amplitude 1396B of the wrapped Gaussian distribution model in the directional depiction 1396. In an example, the model parameter may include a composite parameter, such as a ratio of the positive peak amplitude 1396A to the negative peak amplitude 1396B (also referred to as “max/min ratio”). In another example, the model parameters or features may include amplitude and location of peak 1397A and peak width 1397B of the Gaussian distribution in the longitudinal depiction 1397, representing the mean value (and its spatial location) the standard deviation of the ER features, respectively.

In FIG. 13, and elsewhere in this application, different patterns are illustrated in the figures to represent different color regions. Fewer or more colors may be used. The color heat map may include color gradations. The hotspot view may be layered over an electrode template representing electrodes on the lead. The distribution depictions 1396 and 1397 may include a best fit line for plotted values for the sensed ERs. Various embodiments may enable the user to place bounds around the desired target for the distribution, such as the 1398 bounds on the longitudinal view of the lead. Arrows 1399 or some other indicator may provide a means to identify the largest distribution along the longitudinal lead and the along the segmented electrodes. A user may choose to accept the lead position and/or stimulation setting based on the displayed images on this display (e.g., based on an arrow 1399 being within the bounds 1398).

Various embodiments provide a user interface with data view that allow a user to set an ER target, sense an ER, and provide an indicator (e.g., highlight) whether a given sensed response matches the ER target set by the user. The sensed response target may include a plurality of sensed response values from a plurality of electrodes which may form a distribution of sensed response values.

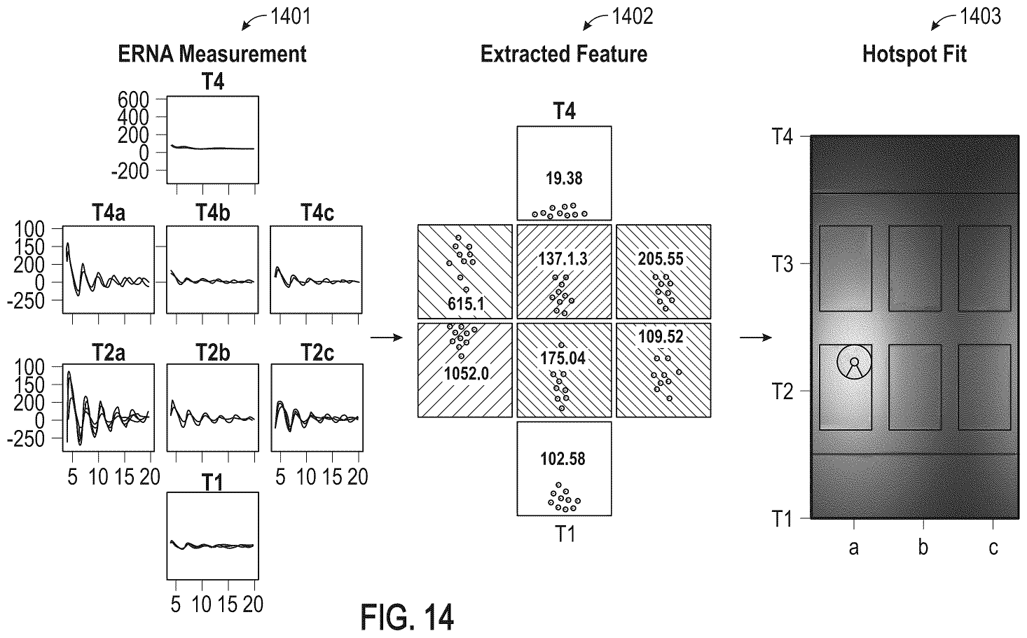

FIG. 14 illustrates, by way of example and not limitation, embodiments for measuring and matching ERs, or ER features or a distribution of ER features derived therefrom, to acceptance criteria such as a template. Each square in the ERNA measurement and the extracted feature views represents an electrode (or electrode segment for the segmented electrodes for the directional lead). The figure illustrates, by way of example and not limitation, two rows of segmented electrodes. The less-processed ERNA measurement is illustrated in the figure at 1401. The ER measurements may be measured at different locations/electrode vertical locations, and directional locations (a, b, c) at a fixed vertical location (e.g., T3a, T3b, T3c). Further multiple measurements may be taken from each set of one or more electrodes in stimulation-ER recording sessions during which stimulation pulses are delivered via a particular stimulating electrode with varying stimulation parameter settings (e.g., stimulation amplitude, frequency, or pulse width).

Feature(s) may be extracted from the raw ERNA measurement data and presented in an extracted feature view 1402. For example, features such as amplitude, magnitude, first peak, width, RMS value, and the like may be extracted from the raw ER signals. Distributions of the extracted features can be generated such as along the longitudinal positions of the lead and/or around angular locations of the segmented electrodes on a circumference of the lead, as shown in depictions 1396 and 1397 in FIG. 13. The extracted features may be used to create sweet spot map or a hotspot fit 1403. The hotspot fit may be color coded similar to thermal mapping, where more features result in a “hotter” color. The hotspot may correspond to a peak of the evoked response (e.g., ERNA) distribution. The measured hotspot may be displayed to the user, as well as an indicator (e.g., highlight) whether the measured response matches the user's or center's ER target. The display to the user may also include fixed or variable offsets to the hotspot. For example, an overlay of the measured ER data (feature distribution), or hotspot, with a visual box of the desired hotspot (i.e., the footprint/template for the institution). A match indicates that the desired or correct location is being stimulation. If there is no match, the ER target provides guidance for moving the electrode or adjusting the stimulation setting until the measured ER (sensed distribution) matches the ER target (target distribution). The desired hotspot or target ER distribution may be dependent on the symptom to be treated (e.g., cognitive or motor functions). For example, an ER target for tremor may be near the STN, and an ER target for bradykinesia may be located higher on the lead. The ER target may be selected from a database (e.g., library) of available targets to provide acceptable distributions in different directions. Some manners of evoking and recording ER may not lend to visualization of the “hotspot” in a two-dimensional (2D) map. This does not preclude the use of the art described herein.

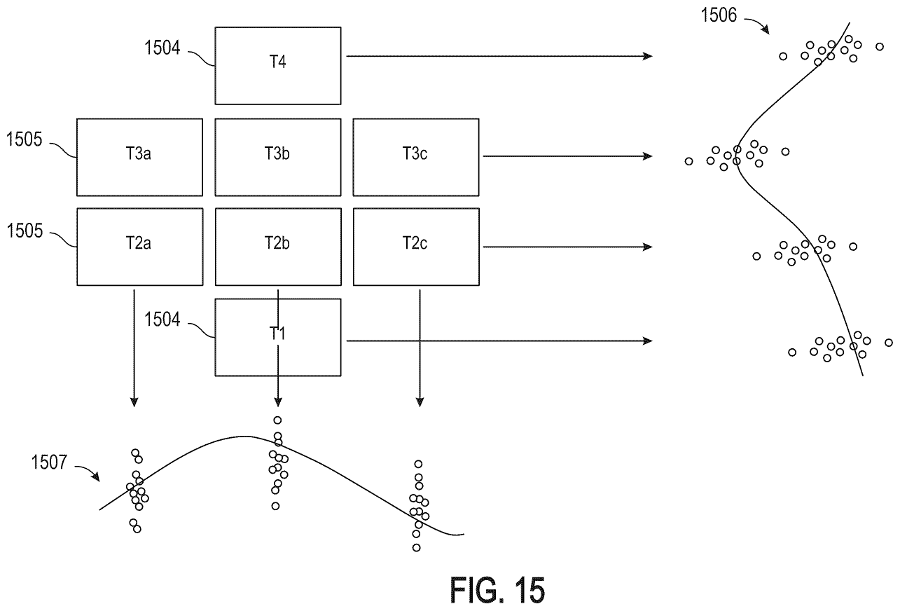

FIG. 15 illustrates, by way of example and not limitation, ER distributions for DBS lead electrodes. The lead may include two ring electrodes 1504 and two segmented electrodes 1505 where each segmented electrode includes three electrodes. The sensed ERs at each electrode are plotted for both the vertical and directional directions. A best fit curve may be created for both the vertical distribution 1506 and directional distribution 1507. In an example, the best fit curve can be determined using a regression analysis. The ER target may include at least a portion of a desired best fit curve in the vertical and/or directional distributions. That is, for example, the best fit curve for the sensed ERs that represents the distribution of the sensed ERs (or ER features) may be compared to the best fit curve for the ER target that represents a target ER distribution.

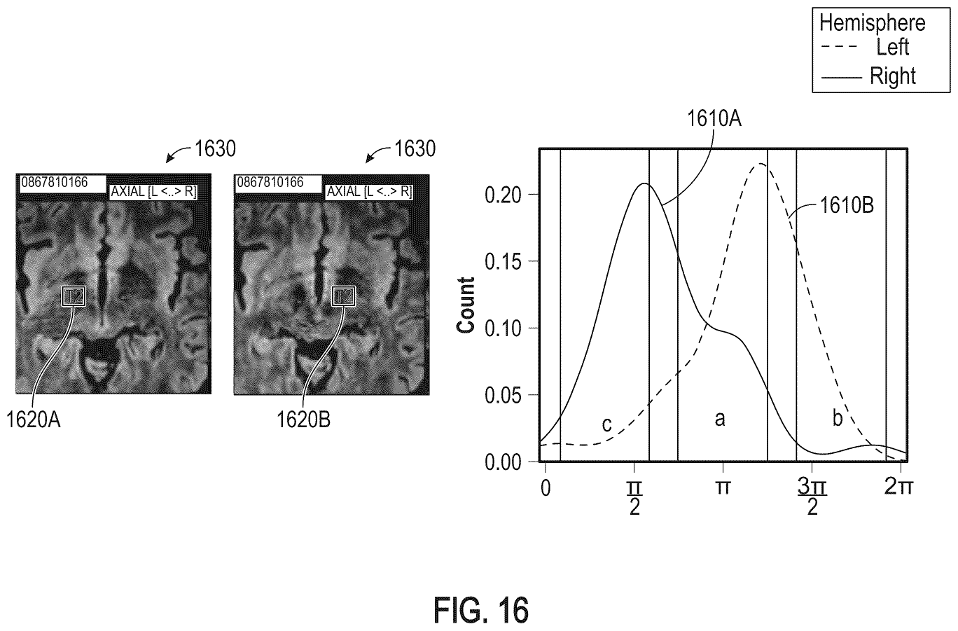

FIG. 16 illustrates, by way of example and not limitation, distributions of ERs sensed respectively from distinct neural targets in left and right hemispheres of a brain. In the illustrated example, left hemisphere ERs can be sensed by multiple segmented electrodes on a DBS lead (such as the row of segmented electrodes 805A or the row of segmented electrodes 805B in FIG. 8, or the segmented electrodes 1505 as shown in FIG. 15) from a left hemisphere target, and right hemisphere ERs can be sensed by multiple segmented electrodes on the DBS lead from a right hemisphere target. The segmented electrodes may be such oriented when a DBS lead is implanted and placed dorsolaterally of the subthalamic nucleus (STN). As discussed above with respect to FIGS. 13-15, a best fit curve or model may be created for directional distribution of the sensed ERs across the segmented electrodes. FIG. 16 illustrates a best fit curve 1610A for the directional distribution of the ERs sensed from the right hemisphere target, and a best fit curve 1610B for the directional distribution of the ERs sensed from the left hemisphere target. The best fit curves 1610A and 1610B are each characterized by a dominant peak. A “hotspot” view that corresponds to the amplitude of the dominant peak in the best fit curve 1610A or the best fit curve 1610B may be created, as similarly discussed above with respect to FIG. 14. As illustrated in FIG. 16, a right hemisphere hotspot 1620A (which corresponds to the peak amplitude of the best fit curve 1610A) and a left hemisphere hotspot 1620B (which corresponds to the peak amplitude of the best fit curve 1610B) may be created and overlay plotted on a radiographic image 1630 of the brain. The best fit curves 1610A and 1610B or features derived therefrom (e.g., peak amplitudes) may be compared to acceptance criteria such as target distribution curve or target ER feature to guide lead placement in a hemisphere or DBS programming. In some examples, the best fit curves 1610A and 1610B may both be used to form a joint left-right hemisphere response curve and compared to a target joint distribution curve or target ER feature to guide lead placement in a hemisphere or DBS programming.

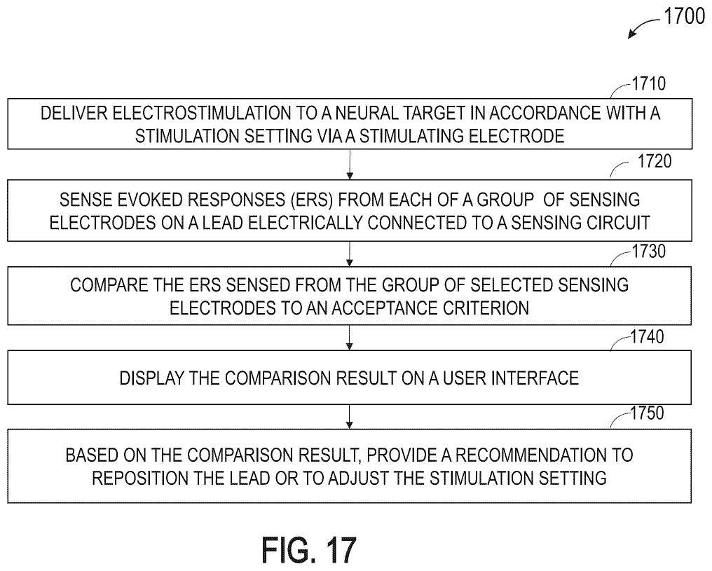

FIG. 17 illustrates, by way of example and not limitation, a method 1700 of guided implantation (e.g., lead placement) and device programming using selected ERs. The method 1700 may be carried out using a medical system such as the neuromodulation system 700. In an example, the method 1700 may be implemented in a programmer device such as RC 45 or CP 50 in communication with an electrostimulator such as IPG 10 or electrostimulator 740. The method 1700 may be used to provide ER-based deep brain stimulation (DBS) at a brain target. The method 1700 may alternatively be used to provide ER-based neuromodulation therapy at other neural targets, such as spinal cord stimulation (SCS) at a spinal neural target.

At 1710, electrostimulation may be delivered to a neural target in accordance with a stimulation setting via a stimulating electrode selected from a plurality of electrodes on at least one lead. At 1720, evoked responses (ERs) may be sensed from each of a group of sensing electrodes electrically connected to a sensing circuit, such as the sensing circuit 710. The group of sensing electrodes can be selected from the plurality of electrodes on the at least one lead. The ERs may be sensed in accordance with a stimulating-sensing electrode configuration, such as the ER sensing map 800 as illustrated in FIG. 8. The stimulating-sensing electrode configuration may include an “on-diagonal” sensing configuration where the same electrode is used for delivering electrostimulation pulses and for recording an ER to that electrostimulation in the same stimulation session. The ER sensed therefrom is referred to as “on-diagonal” ER. The stimulating-sensing electrode configuration may also include an “off-diagonal” sensing configuration where different electrodes are used for delivering electrostimulation pulses and for recording an ER to that electrostimulation in the same stimulation session. The ER sensed therefrom is referred to as “off-diagonal” ER. As described above with respect to FIG. 7, although the “on-diagonal” ERs at the stimulation site may provide useful information about ER distribution and therefore for identifying a match to the desired or target response, the “on-diagonal” ERs are prone to stimulation artifacts and channel saturation, and therefore may introduce errors or not provide usable data and deteriorate the quality of ER-based lead placement and/or device programming. In one example, the stimulating-sensing electrode configuration may include only the “off-diagonal” ERs, but not the “on-diagonal” ERs, in the process of determining a spatial distribution of ERs and for guiding the lead placement and device programming. Alternatively, the stimulating-sensing electrode configuration may be a “nearest neighbor” configuration that includes a selected subset, less than an entirety, of available electrodes for ER sensing. The selected subset can be those non-diagonal electrodes within a specific proximity to the stimulating electrode being used for delivering electrostimulation pulses, examples of which are depicted in FIGS. 9-10. The selective ERs from a subset of sensing electrodes as defined by the stimulating-sensing electrode configuration can improve the efficiency without compromising the accuracy of identifying ERs that match the desired or target response for different desired evoked response targets.

Some sensed ERs, particularly ERs sensed according to the “on-diagonal” configuration or the “nearest neighbor” configuration, may be prone to stimulation artifacts which may contaminate the tissue ER component of interest. Such ERs may be filtered to remove or substantially attenuate the stimulation artifact component. In an example, a parametric model can be generated to fit the stimulation artifact component in the each of the ERs in accordance with a fitting criterion, such as a least-mean squared (LMS) criterion. Examples of the parametric model include an exponential model, a polynomial model, a linear combination (e.g., sum or weighted sum) of exponentials model, or a nonlinear combination of exponentials model. In an example, the parametric model is a polynomial-exponential decay model as given in Equation (1) above. The parametric model fitted artifact component can then be subtracted from the ER to obtain tissue ER of interest, such as the are oscillatory components as illustrated in FIGS. 11A-11D. In some examples, to prevent or reduce model overfitting, parametric model can be generated using a time-reversed version of the raw ER signal to remove or substantially attenuate the artifact component, as described above with respect to FIGS. 12A-12D.

At 1730, the sensed ERs from the group of selected sensing electrodes can be compared to acceptance criteria to produce a comparison result. In an example, a signal feature can be extracted from the sensed ERs, and a comparison can be made between the ER feature to a target ER feature to determine whether a match can be found. Examples of the signal features may include a signal amplitude, magnitude, peak value, value range, a signal curve length, or a signal power or RMS value of an ER signal within a time window, such as the epoch-averaged ERs. The signal amplitude range or value range, also referred to as a peak-to-peak (P2P) value, can be measured as a difference between a maximum value or a minimum value of a dominant peak in the sensed evoked response or an epoch-averaged evoked response within the time window (also referred to as “max P2P” amplitude), or as a difference between a negative peak (trough) and an immediate subsequent positive peak (also referred to as “N1-P2 P2P” amplitude). In some examples, a spatial distribution of the extracted signal features of the sensed ERs across sensing locations of the sensing electrodes can be generated, as illustrated in FIG. 10. A comparison can be made between the spatial distribution of the ER signal features and a target distribution of ER features to determine whether a match can be found.

At 1740, the comparison result can be display on a user interface. Other information, such as one or more of the sensed ERs, the ER features, the spatial distribution of the generated ER features, or the acceptance criterion can also be displayed.

At 1750, based at least in part on the comparison result, a recommendation can be provided to the user to reposition the at least one lead, such as pushing, pulling, shifting, or rotating the lead to achieve a desired target response. The comparison result may additionally or alternatively be used to guide adjustment of stimulation setting. During the repositioning of the at least one lead and/or the adjustment of stimulating setting, the ERs may be sensed, ER features and/or distributions may be determined, and comparison to the accpetance criteria can be updated in substantially real time and displayed to the user. The user may continuate repositioning of the at least one lead and/or adjusting the stimulating setting until the sensed ERs compare more favorably to the acceptance criteria.

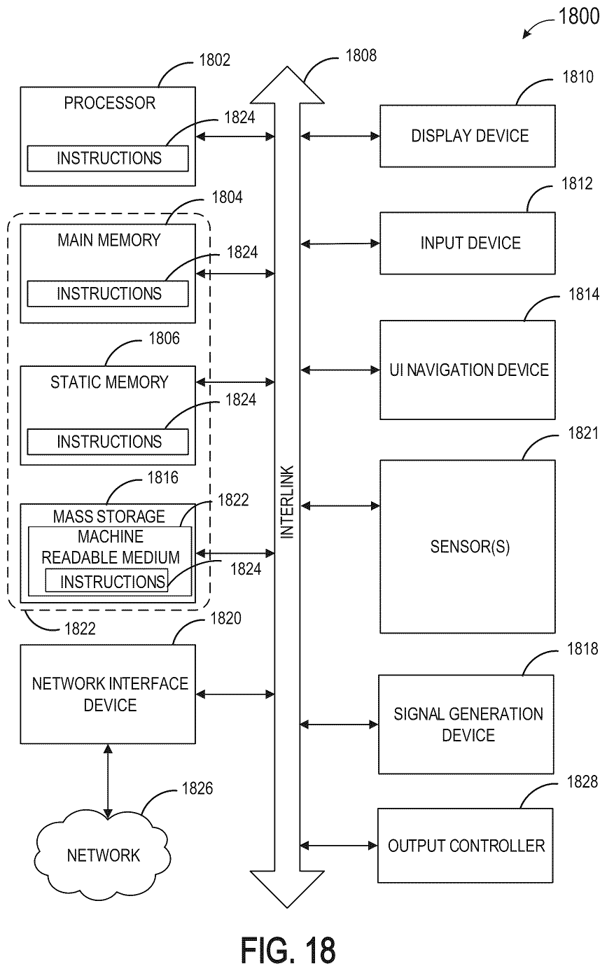

FIG. 18 illustrates generally a block diagram of an example machine 1800 upon which any one or more of the techniques (e.g., methodologies) discussed herein may perform. Portions of this description may apply to the computing framework of various portions of the neuromodulation device or the external programmer device.

In alternative examples, the machine 1800 may operate as a standalone device or may be connected (e.g., networked) to other machines. In a networked deployment, the machine 1800 may operate in the capacity of a server machine, a client machine, or both in server-client network environments. In an example, the machine 1800 may act as a peer machine in peer-to-peer (P2P) (or other distributed) network environment. The machine 1800 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), among other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 1800 may include a hardware processor 1802 (e.g., a central processing unit (CPU), a graphics processing unit (GPU), a hardware processor core, algorithm specific ASIC, or any combination thereof), a main memory 1804 and a static memory 1806, some or all of which may communicate with each other via an interlink (e.g., bus) 1808. The machine 1800 may further include a display unit 1810 (e.g., a raster display, vector display, holographic display, etc.), an alphanumeric input device 1812 (e.g., a keyboard), and a user interface (UI) navigation device 1814 (e.g., a mouse). In an example, the display unit 1810, input device 1812 and UI navigation device 1814 may be a touch screen display. The machine 1800 may additionally include a storage device (e.g., drive unit) 1816, a signal generation device 1818 (e.g., a speaker), a network interface device 1820, and one or more sensors 1821, such as a global positioning system (GPS) sensor, compass, accelerometer, or other sensors. The machine 1800 may include an output controller 1828, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 1816 may include a machine-readable medium 1822 on which is stored one or more sets of data structures or instructions 1824 (e.g., software) embodying or utilized by any one or more of the techniques or functions described herein. The instructions 1824 may also reside, completely or at least partially, within the main memory 1804, within static memory 1806, or within the hardware processor 1802 during execution thereof by the machine 1800. In an example, one or any combination of the hardware processor 1802, the main memory 1804, the static memory 1806, or the storage device 1816 may constitute machine readable media.

While the machine-readable medium 1822 is illustrated as a single medium, the term “machine readable medium” may include a single medium or multiple media (e.g., a centralized or distributed database, and/or associated caches and servers) configured to store the one or more instructions 1824.

The term “machine readable medium” may include any medium that is capable of storing, encoding, or carrying instructions for execution by the machine 1800 and that cause the machine 1800 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EPSOM)) and flash memory devices; magnetic disks, such as internal hard disks and removable disks; magneto-optical disks; and CD-ROM and DVD-ROM disks.

The instructions 1824 may further be transmitted or received over a communication network 1826 using a transmission medium via the network interface device 1820 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 1820 may include one or more physical jacks (e.g., Ethernet, coaxial, or phone jacks) or one or more antennas to connect to the communication network 1826. In an example, the network interface device 1820 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1800, and includes digital or analog communications signals or other intangible medium to facilitate communication of such software.

Various examples are illustrated in the figures above. One or more features from one or more of these examples may be combined to form other examples.

The method examples described herein may be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code may form portions of computer program products. Further, the code may be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical-device system, comprising:
at least one lead including a plurality of electrodes;
an electrostimulator configured to provide electrostimulation to a neural target of a patient;
a sensing circuit configured to sense an evoked response (ER) to the electrostimulation; and
a controller circuit operably connected to the electrostimulator and the sensing circuit, the controller circuit configured to:
deliver the electrostimulation to the neural target in accordance with a stimulation setting via a stimulating electrode selected from the plurality of electrodes on the at least one lead;
collect sensed ERs to the electrostimulation from each of a group of sensing electrodes selected from, and less than an entirety of, the plurality of electrodes on the at least one lead, the group of selected sensing electrodes located within a specific proximity to the selected stimulating electrode;
compare the ERs sensed from the group of selected sensing electrodes to an acceptance criterion to produce a comparison result; and
display the ERs and the comparison result on a user interface.

2. The medical-device system of claim 1, wherein the selected group of sensing electrodes include two or more electrodes immediate adjacent to the stimulating electrode on the at least one lead.

3. The medical-device system of claim 1, wherein the controller circuit is further configured to, based at least in part on the comparison result, provide a recommendation on the user interface to reposition the at least one lead or to adjust the stimulation setting to cause the sensed ERs from the group of selected sensing electrodes to compare more favorably to the acceptance criterion.

4. The medical-device system of claim 1, wherein the at least one lead includes a deep brain stimulation (DBS) lead, and wherein the electrostimulator is configured to provide DBS to a brain target of the patient.

5. The medical-device system of claim 1, wherein the plurality of electrodes include one or more ring electrodes disposed at respective longitudinal positions along a length of the at least one lead, or one or more rows of segmented electrodes where each row comprises segmented electrodes disposed about a circumference of the at least one lead at a specific longitudinal position,
wherein the stimulating electrode and the group of selected sensing electrodes are each selected from the one or more ring electrodes or the one or more rows of segmented electrodes.

6. The medical-device system of claim 1, wherein the acceptance criterion includes a target distribution of ERs across the group of selected sensing electrodes,
wherein the controller circuit is configured to:
determine a spatial distribution of the sensed ERs across the group of selected sensing electrodes; and
provide a recommendation on the user interface to reposition the at least one lead or to adjust the stimulation setting based at least in part on a comparison of the determined spatial distribution of the sensed ERs and the target distribution.

7. The medical-device system of claim 1, wherein the acceptance criterion includes a target ER feature,
wherein the controller circuit is configured to:
determine an ER feature from the ERs sensed from the group of selected sensing electrodes; and
provide a recommendation on the user interface to reposition the at least one lead or to adjust the stimulation setting based at least in part on a comparison of the determined ER feature to the target ER feature.

8. The medical-device system of claim 1, wherein the controller circuit is further configured to filter the ERs sensed from the group of selected sensing electrodes to remove or substantially attenuate an artifact component from each of the ERs, and to compare the filtered ERs to the acceptance criterion to produce the comparison result.

9. The medical-device system of claim 8, wherein to filter the ERs sensed from the group of selected sensing electrodes, the controller circuit is configured to, for each of the ERs:
generate a parametric model to fit the artifact component in the each of the ERs in accordance with a fitting criterion; and
subtract the parametric model fitted artifact component from the each of the ERs.

10. The medical-device system of claim 9, wherein the parametric model is a polynomial-exponential decay model.

11. The medical-device system of claim 8, wherein to filter the ERs sensed from the group of selected sensing electrodes, the controller circuit is configured to, for each of the ERs:
generate a time-reversed signal for each of the ERs;
generate a parametric model to fit the artifact component in the time-reversed signal in accordance with a fitting criterion; and
subtract the parametric model fitted artifact component from the time-reversed signal.

12. The medical-device system of claim 1, wherein the controller circuit is configured to intermittently pause delivery of the electrostimulation, and to collect the ERs sensed from the group of selected sensing electrodes during the intermittent pause.

13. A method of providing neurostimulation to a neural target of a patient via a medical-device system that comprises an electrostimulator and at least one lead coupled thereto, the method comprising:
delivering electrostimulation to the neural target in accordance with a stimulation setting via a stimulating electrode selected from a plurality of electrodes on the at least one lead;
sensing evoked responses (ERs) from each of a group of sensing electrodes electrically connected to a sensing circuit, the group of sensing electrodes selected from, and less than an entirety of, the plurality of electrodes on the at least one lead, and located within a specific proximity to the selected stimulating electrode;
comparing the ERs sensed from the group of selected sensing electrodes to an acceptance criterion to produce a comparison result;
displaying the ERs and the comparison result on a user interface; and
based at least in part on the comparison result, providing a recommendation on the user interface to reposition the at least one lead or to adjust the stimulation setting to cause the sensed ERs from the group of selected sensing electrodes to compare more favorably to the acceptance criterion.

14. The method of claim 13, wherein the selected group of sensing electrodes include two or more electrodes immediate adjacent to the stimulating electrode on the at least one lead.

15. The method of claim 13, comprising determining a spatial distribution of the sensed ERs across the group of selected sensing electrodes,
wherein the recommendation to reposition the at least one lead or to adjust the stimulation setting is based at least in part on a comparison of the determined spatial distribution of the sensed ERs and a target distribution of ERs across the group of selected sensing electrodes.

16. The method of claim 13, comprising determining an ER feature from the ERs sensed from the group of selected sensing electrodes,
wherein the recommendation to reposition the at least one lead or to adjust the stimulation setting is based at least in part on a comparison of the determined ER feature to a target ER feature.

17. The method of claim 13, comprising filtering the ERs sensed from the group of selected sensing electrodes to remove or substantially attenuate an artifact component from each of the ERs, and comparing the filtered ERs to the acceptance criterion to produce the comparison result.

18. The method of claim 17, wherein filtering the ERs sensed from the group of selected sensing electrodes includes, for each of the ERs:
generating a parametric model to fit the artifact component in the each of the ERs in accordance with a fitting criterion; and
subtracting the parametric model fitted artifact component from the each of the ERs.

19. The method of claim 18, wherein the parametric model is a polynomial-exponential decay model.

20. The method of claim 17, wherein filtering the ERs sensed from the group of selected sensing electrodes includes, for each of the ERs:
generating a time-reversed signal for each of the ERs;
generating a parametric model to fit the artifact component in the time-reversed signal in accordance with a fitting criterion; and
subtracting the parametric model fitted artifact component from the time-reversed signal.
